(19) United States
(12) Patent Application Publication
(10) Pub. No.: US 2018/0036557 A1
(43) Pub. Date: Feb. 8, 2018

(54) RADIATION THERAPY APPARATUS, TREATMENT PLANNING DEVICE, AND METHOD FOR CONTROLLING POSITION OF RADIATION THERAPY APPARATUS

(71) Applicant: Accuthera Inc., Kawasaki-city (JP)

(72) Inventors: Eiki Yoshimizu, Tokyo (JP); Takashi Kiuchi, Yokohama (JP)

(73) Assignee: Accuthera Inc., Kawasaki-city (JP)

(21) Appl. No.: 15/667,627

(22) Filed: Aug. 3, 2017

(30) Foreign Application Priority Data
Aug. 8, 2016 (JP) 2016-155849

(51) Int. Cl. A61N 5/10

(52) U.S. Cl. CPC A61N 5/1083; A61N 5/1037; A61N 5/1049; A61N 2005/1089

(57) ABSTRACT

A radiation therapy apparatus includes a manipulator; a radiation head; a correction unit; and first and second controllers. The manipulator includes an arm movable about n axes (where n is 6 or more). The radiation head, supported by the arm, is configured to emit radiation. The correction unit continuously corrects positional coordinates of the radiation head on the manipulator so that radiation emitted from the radiation head is directed onto a reference point while the radiation head is moved along a predetermined route on a virtual sphere having the reference point as its center. The first controller controls the manipulator so that the radiation head is moved along the route using the corrected positional coordinates. The second controller causes the radiation head to emit radiation toward the reference point as the radiation head passes through radiation emission points on the route.

### CROSS-REFERENCE TO RELATED APPLICATION

The present application claims priority pursuant to 35 U.S.C. §119(a) from Japanese patent application number 2016-155849, filed on Aug. 8, 2016 in the Japan Patent Office, the entire disclosure of which is hereby incorporated by reference herein.

### BACKGROUND

### Technical Field

The present disclosure relates to a radiation therapy apparatus using a robot manipulator, in particular, to a method of controlling movement of a radiation emitting head of the radiation therapy apparatus, and to a treatment planning device used by the radiation therapy apparatus.

### Related Art

In radiation therapy (for example, X-ray treatment), it is desirable to accurately irradiate abnormal cells, such as tumors, with X-rays, but not to irradiate healthy cells as much as possible. Thus, radiation is performed at a lower dose from multiple directions, thereby minimizing the effects on healthy cells. As one example of such multidirectional radiation, known is a method of using a multi-axis robot manipulator to move a radiation head along a virtual spherical surface having a predetermined radius about a treatment reference point (isocenter). A robot arm moves the radiation head to a space node at which radiation at a given dose is administered in accordance with a specified route.

In conventional multidirectional radiation, the radiation head is stopped at each node to perform radiation. When radiation at one node is finished, the radiation head is moved to the next node and stopped again to perform radiation. The conventional multidirectional radiation requires not only a long time for moving along a radiation route, but also time for stabilizing vibrations associated with acceleration and deceleration before and after stopping the radiation head. This approach necessitates a lengthy treatment time for each treatment session. Accordingly, it is preferable to perform radiation at radiation emission points while moving the radiation head in a continuous manner to reduce the time needed for treatment.

### SUMMARY

When the radiation head is driven with the multi-axis robot manipulator, the radiation head held at the distal end of the arm assumes a complex posture at each node. Flexure is generated in each posture including flexure generated by the radiation head's own weight, and the radiation head may deviate from the reference coordinates held by the robot manipulator. In the conventional method, the radiation head is stopped at each node to correct position deviation caused by such flexure.

The inventors have found that, in a case where radiation is performed while the radiation head is moved, a deviation is generated between positional coordinates corrected in a stopped state and a radiation emission position during movement.

To avoid this problem, the present disclosure describes a technique for controlling movement of a radiation head that provides quick and accurate radiation emission.

That is, a radiation head performs radiation when passing through a radiation emission point on a route while the radiation head is continuously moved along a radiation route that has been subjected to dynamic position correction.

In one aspect of this disclosure, an improved radiation therapy apparatus includes: a manipulator including an arm movable about n axes (where n is a natural number of 6 or more); a radiation head to emit radiation while being supported by the arm; and a correction unit to correct positional coordinates of the radiation head on the manipulator so that radiation emitted from the radiation head is directed onto a reference point while the radiation head is moved along a predetermined route on a virtual sphere S having the reference point as its center, a first controller; and a second controller. The first controller controls the manipulator so that the radiation head is moved along the route using corrected positional coordinates. The second controller causes the radiation head to emit radiation toward the reference point as the radiation head passes through radiation emission points on the route.

With the above-described configuration, quick and accurate radiation emission can be performed.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is an external view illustrating a radiation therapy apparatus to which the present disclosure is applied.

FIG. 1B is a diagram illustrating an example of correction for positional coordinates of a six-axis manipulator using a correction jig.

FIG. 2A is a schematic diagram illustrating a radiation therapy procedure.

FIG. 2B is a system block diagram illustrating a radiation therapy system.

FIGS. 3A to 3C are diagrams illustrating dynamic corrections of positional coordinates of an X-ray head.

FIG. 4 is a diagram illustrating an example of a standard route as an optimal radiation route.

FIGS. 5A to 5E are diagrams illustrating various patterns of a standard route.

FIGS. 6A to 6C are diagrams illustrating methods of changing direction of a radiation route.

FIG. 7 is a diagram illustrating arrangement of auxiliary nodes.

FIG. 8 is a diagram illustrating setting of standard radiation regions.

FIGS. 9A and 9B are diagrams illustrating setting of radiation regions with interference being avoided.

FIG. 10 is a diagram illustrating setting of spare nodes.

FIG. 11 is a flow chart illustrating an operation example of a radiation therapy apparatus using spare nodes.

FIG. 12 is a diagram illustrating a hardware configuration of a controller disposed within a manipulator.

FIG. 13 is a functional block diagram illustrating a controller disposed within a manipulator.

FIG. 14 is a diagram illustrating a hardware configuration of a therapy apparatus.

FIG. 15 is a functional block diagram illustrating a therapy apparatus.

FIG. 16 is a schematic diagram illustrating a principal part of an X-ray head.

FIG. 17 is a diagram illustrating the outline of a control system configured to control swinging movement of a collimator device.

FIG. 18 is a configuration diagram illustrating a principal part for controlling swinging movement of a collimator device.

FIG. 19 illustrates generation of control outputs for performing swinging movement of a swing collimator.

FIG. 20 is a perspective view illustrating an X-ray radiation apparatus.

FIG. 21 is a perspective view illustrating another configuration example of a collimator device.

FIG. 22 is a diagram illustrating an X-axis and a Y-axis serving as rotational axes of swinging movement.

FIG. 23 is a side view illustrating a collimator device.

### DETAILED DESCRIPTION

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments shown in the drawings, specific terminology is employed for the sake of clarity. However, the present disclosure is not intended to be limited to the specific terminology so selected and it is to be understood that each specific element includes all technical equivalents that have the same function, operate in a similar manner, and achieve a similar result.

In the present disclosure, radiation is performed when a radiation head passes through each of radiation emission points (nodes) without being stopped at each of the radiation emission points, in contrast to conventional common irradiation methods. Thus, positional correction is required so as to pass through precise radiation positions while the radiation head is being moved. Prior to the detailed description of an embodiment, general points to be considered in implementing radiation and position correction during movement of the radiation head will be described.

(1) In a case where the radiation head performs radiation while being continuously moved with a robot arm, such as a six-axis manipulator, when passing through a node, variations in radiation position and unevenness in radiation accuracy increase, in contrast to conventional stop radiation. Not only variations depending on nodes, but even at the same node, positional errors vary according to the direction in which the radiation head is moved on a virtual sphere S, i.e., vertical direction (direction of longitudinal line), lateral direction (direction of latitudinal line), direction of gravity, or direction opposite to gravity. This is a new issue to be considered in performing radiation emission during continuous movement.

(2) Assuming that a point of intersection between a latitudinal line and a longitudinal line on a virtual sphere S is a radiation emission point (i.e., node), an actual movement distance is different between the vicinity of the poles and the vicinity of the equator on the virtual sphere S, even with regular angular intervals. An aspect of multidirectional radiation is to perform radiation from different positions on the virtual sphere S, to minimize the effects on healthy cells. Accordingly, intervals of distance between nodes are more important than angular intervals. When considering the maximum radiation field, it is necessary to secure radiation emission points that do not overlap on the virtual sphere S.

(3) In order to minimize a time period for stabilizing vibrations associated with acceleration and deceleration before/after stopping the radiation head, it is preferable that the radiation head minimizes change in speed without being stopped, even when changing direction along a radiation route.

(4) If many changes in direction are included along the radiation route, a deviation is generated in a robot manipulator due to centrifugal force and/or its posture at the time of change in direction, and vibrations may be generated in some cases. Accordingly, it is necessary to create a radiation route that minimizes the possibility of generation of such vibrations.

(5) In view of efficiency of position correction, it is preferable to formulate, as a standard treatment route, an optimal radiation route satisfying at least a part of the above-described consideration points (2) to (4).

(6) If a movement route for the radiation head has an unlimited degree of freedom, position correction should be minutely controlled for each patient, resulting in increase in workload. Furthermore, creation of a treatment plan becomes difficult. Thus, it is preferable to formulate a radiation route within a reasonable range.

(7) When the radiation head performs radiation while being moved, there may be a case where radiation cannot be performed at a node or a case where it is better not to perform radiation. Thus, measures to be taken concerning these cases are necessary. The case where radiation cannot be performed is, for example, a case where a patient moves due to a cough or a sneeze. The case where it is better not to perform radiation is generation of a signal error, a failure to read position detection , fail-safe at the time of malfunction detection, or the like.

In order to deal with the consideration point (1), the position of the radiation head in the coordinate system of the six-axis manipulator is subjected to dynamic position correction. Here, the term “dynamic position correction” indicates correction of a position deviation generated in the continuous movement of the radiation head. In a case of radiation during movement, a position deviation that deviates from a position correction value obtained in a stopped state may be generated. Corrected positional coordinates are used to move the radiation head, thereby enhancing accuracy in position and radiation when emitting radiation during movement. In addition, positional accuracy in irradiation during movement can further be enhanced such that position correction is individually performed according to the direction of movement of the radiation head. In view of reduction in variations and correction time, a standard route of the radiation head is set on the virtual sphere S, and position correction is performed at a predetermined speed. A speed when performing position correction is set at a speed, for example, in a range from ¼ to 1 times the movement speed of the radiation head.

In order to deal with the consideration point (2), one or more auxiliary nodes are provided between adjacent nodes in low and middle latitudes regions on the virtual sphere S. This arrangement thus provides nodes at regular intervals along the radiation route, thereby implementing efficient radiation.

In order to deal with the consideration point (3), the radiation head is to continue being moved without being stopped when changing direction along a radiation route. This minimizes generation of vibrations, reduces treatment time, and further enables stable position correction.

In order to deal with the consideration point (4), radiation routes that include a change in direction are avoided as much as possible. More specifically, a route having fewer curves and longer straight parts is desirable. For example, a route including nodes continuing parallel to a latitudinal line or a longitudinal line is desirable. This enables a radiation route including minimum change in direction and many nodes.

In order to deal with the consideration point (5), a route having fewer curves which can be drawn with a continuous single stroke or a route having fewer interference elements is set as a standard route. Setting of several standard patterned routes can enhance efficiency of dynamic correction.

In order to deal with the consideration point (6), one or more standard patterned routes are prepared for each treatment region, and an optimal standard route is used for each patient. This can reduce variations in route and treatment time. Further, creation of a treatment plan is facilitated.

In order to deal with the consideration point (7), one or more spare nodes are included in the patterned radiation route. Even if a node at which radiation cannot be performed is generated during treatment, such a node is skipped and radiation is performed at a spare node located next to the skipped node, so that treatment can be continued.

Hereinafter, an embodiment will be described in detail with reference to drawings.

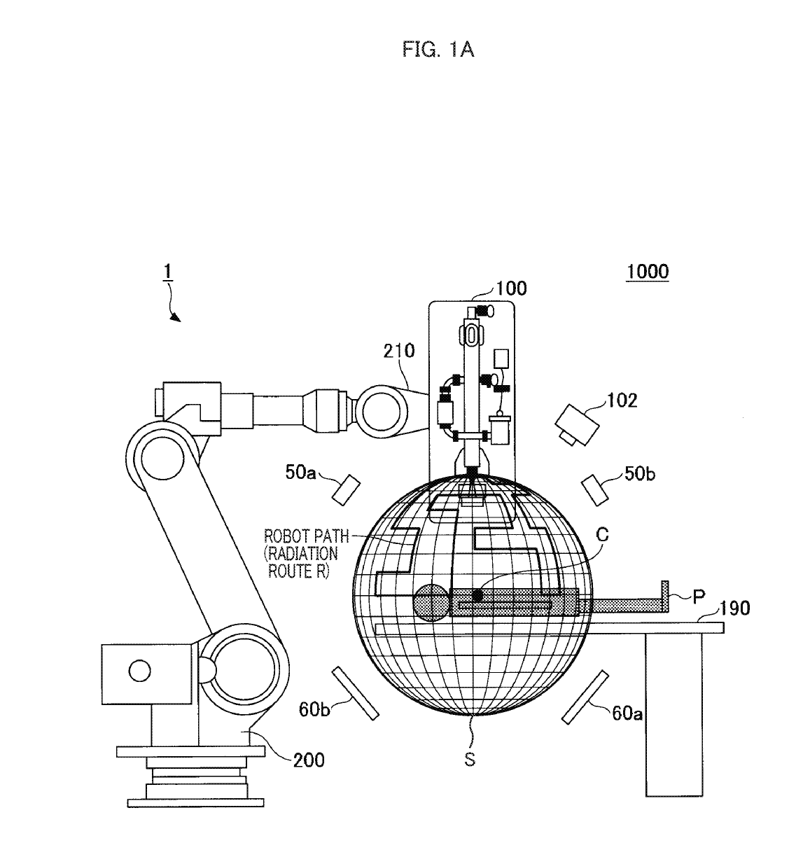

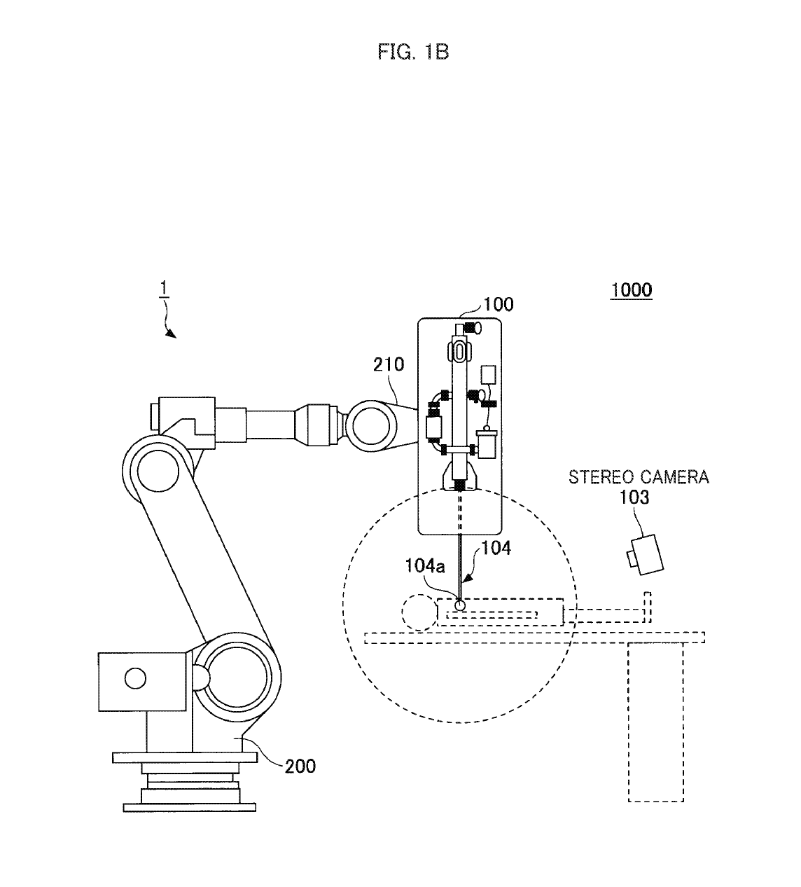

FIG. 1A is a schematic diagram illustrating a radiation therapy system 1000 to which control of movement of a radiation head according to the present disclosure is applied. The radiation therapy system 1000 comprises a radiation therapy apparatus 1, an imager using sets of X-ray tubes 50a, 50b and detectors 60a, 60b, and a body surface monitoring camera 102. The detectors 60a, 60b are, for example, Flat Panel Detectors (FPDs), which are flat X-ray detectors. The body surface monitoring camera 102 captures motion of a body surface near an affected area. Information on motion of a body surface is used for setting of initial parameters for the radiation therapy apparatus 1, together with information on heartbeat and breathing phase.

The radiation therapy apparatus 1 comprises a six-axis manipulator 200 and an X-ray head 100 connected to an end of an arm 210 of the six-axis manipulator 200. The six-axis manipulator 200 is one example of a robot manipulator, and the robot manipulator having six axes or more may be used. The X-ray head 100 is one example of the radiation head, and radiation may be gamma radiation or uncharged particle radiation (neutron radiation, etc.). The arm 210 is capable of moving along three axes as well as rotationally about the axes, and causes the X-ray head 100 to assume a given posture. When a patient is subjected to X-ray therapy while lying on a couch 190, the six-axis manipulator 200 directs the X-ray head 100 in a desired direction while moving the X-ray head 100 along a radiation route R over a virtual sphere S.

The robot-type radiation therapy apparatus 1, which includes the six-axis manipulator 200 mounting the X-ray head 100, omnidirectionally irradiates an affected area from any point on the virtual sphere S. The X-ray head 100 moves on the virtual sphere S having a predetermined radius from an isocenter C such that X-ray radiation is directed, at all times, toward the isocenter C, which is an irradiation region, while maintaining a constant Source Axis Distance (SAD). The SAD indicates a distance from an X-ray source to the isocenter C and, for example, 600 mm is set for a head, while 800 mm is set for a torso.

In an embodiment, X-ray radiation is performed when the X-ray head 100 passes through radiation emission points (nodes) set along the radiation route R without being stopped thereat. The positional coordinates of the X-ray head 100 are corrected also during its continuous movement so that the X-ray head 100 passes through these precise positions. This configuration eliminates the need for a time period for stabilizing vibrations during a standstill of the X-ray head 100 and vibrations generated in association with acceleration and deceleration before/after the standstill, thereby achieving quick and accurate irradiation.

FIG. 1B is a diagram illustrating an example of position correction for the X-ray head 100. The positional coordinates of the X-ray head 100 are corrected also during its continuous movement in advance of a treatment, so that a radiation field is directed at all times toward the isocenter, i.e., a reference point of the treatment, and the constant SAD is maintained (dynamic position correction). The dynamic position correction may be performed using, for example, a stereo camera 103 and a correction tool 104. The correction tool 104 is a stiff, lightweight rod-shaped jig with a small sphere 104a at its end. The correction tool 104 has the small sphere 104a at the position of the isocenter, and is mounted to a radiation head 100 such that the center axis of the correction tool 104 is aligned with the X-ray radiation axis of the radiation head 100. The correction tool 104 is manufactured such that, even if the direction of the X-ray head 100 set by the six-axis manipulator 200 is changed, the small sphere 104a is located at the position of the isocenter position (position having the SAD from the X-ray source) without occurrence of curve or deviation.

The six-axis manipulator 200 is operated such that an emission opening of the X-ray head 100 is directed toward the isocenter, as well as the radiation source within the X-ray head 100 is continuously moved along the virtual sphere S having a radius of the SAD about the small sphere 104a located at the position of the isocenter. At this time, the small sphere 104a is imaged using the stereo camera 103, and the center coordinates (isocenter coordinates) of the small sphere 104a are acquired through image processing. A position deviation from the isocenter, i.e., the center of the small sphere 104a, can be calculated at each of the radiation emission points (nodes), and data obtained as such is used as a correction value. With one or multiple such passes, substantially no movement of the small sphere 104a is caused by an operation of the manipulator 200 after correction. In other words, irrespective of the position and/or direction of the X-ray head 100, the emission opening of the X-ray head 100 on the virtual sphere S is directed toward the isocenter at all times. At this point correction is finished, and the correction tool 104 is demounted. The final positional coordinates of the X-ray head 100 when the correction is finished are recorded as a correction value.

Such dynamic position correction to the six-axis manipulator can be performed at an appropriate frequency, such as before shipment of the radiation therapy apparatus 1, at the time of delivery (mounting) thereof, once a month, once a week, once a day, or with every patient.

The radiation therapy apparatus 1 can track motion of an affected area, in addition to controlling movement of the X-ray head 100. When a radiation target is a respiratory or cardiac tumor, such a tumor itself moves in a complex manner, accelerating and decelerating in association with breathing and heartbeat of a patient. Thus, using a swing collimator disposed within the X-ray head 100, an irradiation spot is caused to track motion of the lungs or the like. Configuration and operation of the swing collimator will be described later in detail.

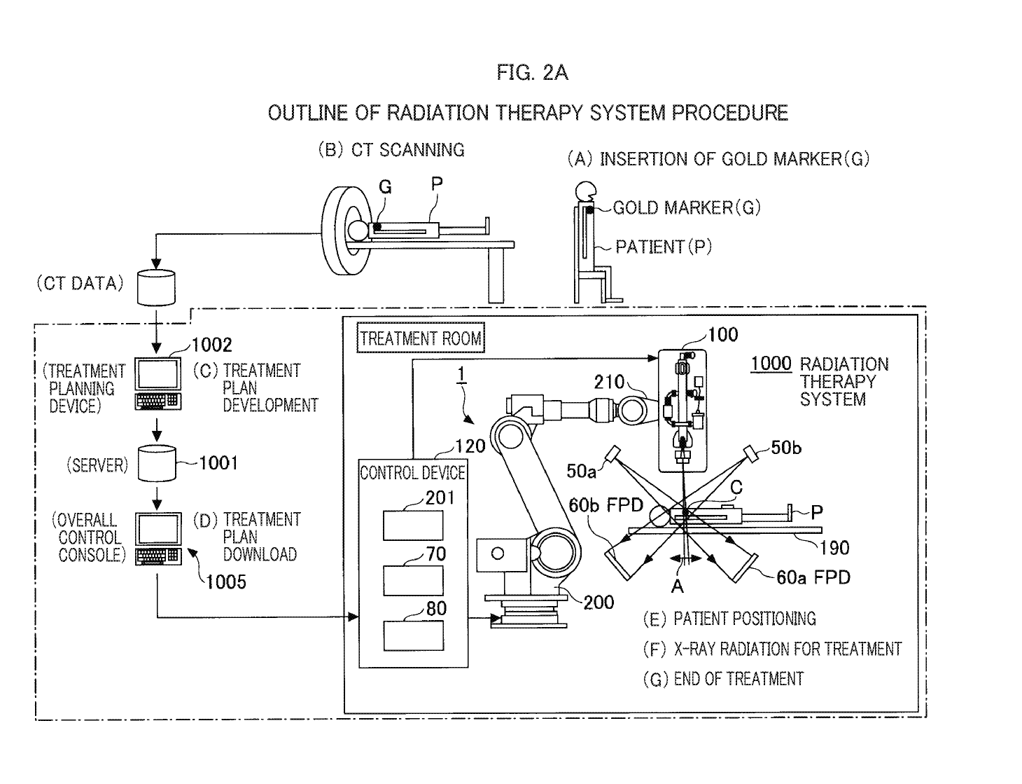

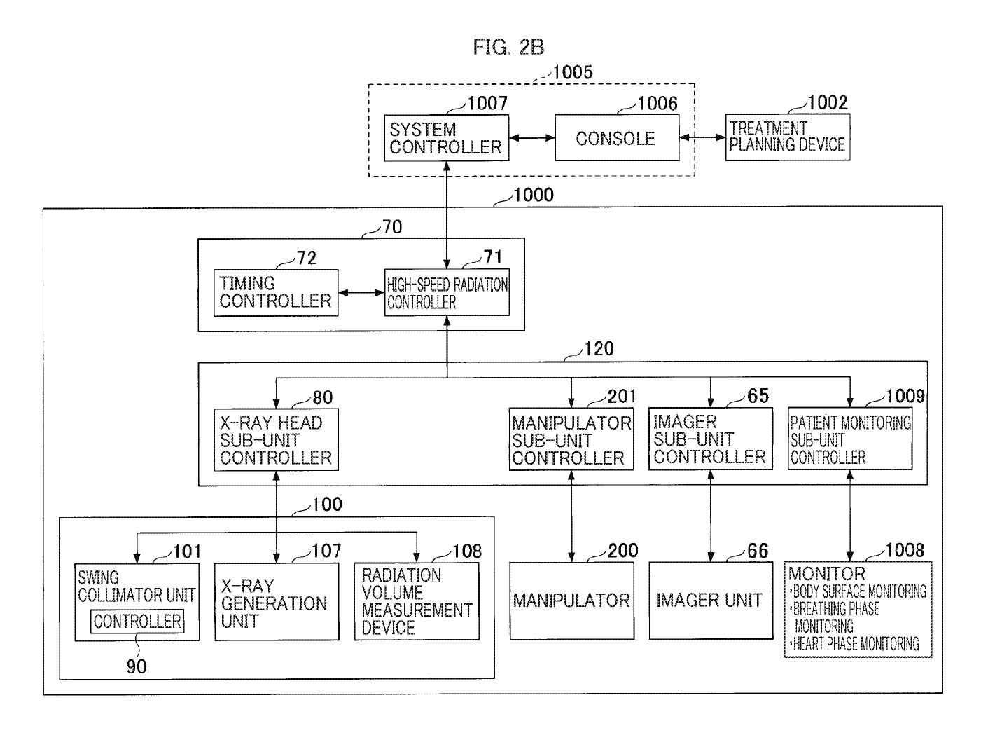

FIG. 2A is a schematic diagram illustrating a radiation therapy procedure using the radiation therapy system 1000. The processes (A) to (G) describe the outline of the procedures of the radiation therapy.

(A) A single or a plurality of markers is implanted near an affected area of a patient P who is subjected to radiation therapy. The marker is made of a material that attenuates radiation and, for example, a gold marker G is used. The single gold marker G is illustrated for the sake of convenience, but a plurality of markers may be used. The gold marker G has a spherical body having a diameter of about 1.5 mm, and is utilized to specify the affected area in an X-ray image.

(B) After it is confirmed that the gold marker G has been fixed, the patient P is subjected to Computer Tomography (CT) scanning with a CT scanner to obtain CT scan data.

(C) A physician or the like operates a treatment planning device 1002 and creates an X-ray treatment plan for the patient P based on the CT scan data. Specifically, a Region of Interest (ROI: image region of interest) in the affected area and target dose distribution are specified. The optimum direction of irradiation, dose, and radiation route are calculated using treatment plan software. In an embodiment, the radiation route is a route on which the X-ray head 100 moves at a constant speed along a predetermined route on the virtual sphere S. The term “X-ray treatment plan” means a plan of direction/dose, etc., for irradiating the affected area of the patient P with X-rays.

(D) An operator downloads the data of the created treatment plan from a server 1001 to an overall control console 1005 of the radiation therapy apparatus 1.

(E) Lay and position the patient P on the couch.

(F) An operator operates the radiation therapy apparatus 1 so as to irradiate the patient P with X-rays for treatment.

(G) The treatment is finished, and the patient P is set down from he couch and is allowed to exit from the treatment room.

In the above-described process (F), X-ray radiation is performed at an optimized dose and in an optimized direction according to the radiation route created by the treatment planning device. More specifically, the X-ray head 100 is driven with the six-axis manipulator 200 along the designated radiation route. At this time, the X-ray head 100 is continuously moved according to the positional coordinates corrected in advance, and performs X-ray radiation when passing through nodes.

On the other hand, the position of the gold marker G is detected by an imager device that includes the X-ray tubes 50a, 50b and the FPDs 60a, 60b, and the position of a tumor is calculated based on CT information imaged in advance. The swing collimator mounted to the X-ray head 100 is used to direct a radiation field toward the position of the tumor that is obtained by calculation, to perform X-ray radiation.

Assuming that the X-ray head 100 has a weight of about 200 kg, it is difficult to make the X-ray head 100 itself track motion of the lungs using the six-axis manipulator 200, in view of a typical breathing period (about 0.5 Hz) and heartbeat (about 1 Hz). This is because the six-axis manipulator 200 has a resonance frequency of about 4 Hz, and a crossover frequency (about 2 Hz) for control cannot be raised. With this crossover frequency, sufficient positional accuracy cannot be acquired.

In an embodiment of the present disclosure, the six-axis manipulator 200 is operated so that the X-ray head 100 is directed at all times towards the isocenter as well as accurately moved at a predetermined speed through the nodes on the virtual sphere S. Motion of the lungs and the heart is tracked using only the swing control of the swing collimator having a small mass. Accordingly, it is possible to have a great crossover frequency, thereby improving position tracking accuracy.

The radiation therapy apparatus 1 further comprises a control device 120 to control the overall operation of the radiation therapy apparatus 1. The control device 120 includes an overall control unit 70, an X-ray head sub-unit controller 80, and a manipulator sub-controller 201. A detailed description of functions and operations of these controllers is deferred.

FIG. 2B is a system block diagram illustrating the radiation therapy system 1000. The radiation therapy system 1000 is connected to the overall control console 1005. The overall control console 1005 comprises: a console 1006 including a display device and an input device; and a system controller 1007. A treatment planning device 1002 is connected to the overall control console 1005.

In an example of FIG. 2B, the overall control unit 70 is illustrated outside the control device 120, but may be provided inside the control device 120. The overall control unit 70 comprises a high-speed radiation controller 71 and a timing controller 72. The control device 120 comprises the X-ray head sub-unit controller 80, a manipulator sub-unit controller 201, an imager sub-unit controller 65, and a patient monitoring sub-unit controller 1009. The X-ray head sub-unit controller 80 controls the X-ray head 100. The manipulator sub-unit controller 201 controls the six-axis manipulator 200. The imager sub-unit controller 65 controls an imager 66 including the X-ray tubes 50a, 50b and the FPDs 60a, 60b. The patient monitoring sub-unit controller 1009 controls a monitor set including the body surface monitoring camera 102. The X-ray head 100 comprises a swing collimator unit as a collimator device 101, an X-ray generation unit 107, and a radiation volume measurement device 108, as will be described later.

The six-axis manipulator 200 continuously drives the X-ray head 100 along the radiation route that has been subjected to dynamic position correction by control of the manipulator sub-unit controller 201. The X-ray head 100 performs X-ray radiation toward the isocenter during its movement, when passing through a predetermined radiation emission point, under control of at least the X-ray head sub-unit controller 80.

Multidirectional dynamic tracking radiation is implemented through the movement on the virtual sphere S targeting the isocenter using the six-axis manipulator 200 and through the movement of the collimator device (swing collimator) 101 tracking motion of a tumor. Dynamic detection is performed by the imager 66. Estimated position is calculated by the high-speed radiation controller 71, using imager information during treatment and manipulator movement information based on position information obtained from a treatment plan. The estimated position is provided to the collimator device 101 so that the collimator device 101 performs dynamic tracking. X-ray radiation timing is controlled based on the radiation position in the six-axis manipulator 200 that has been subjected to dynamic position correction according to the treatment plan.

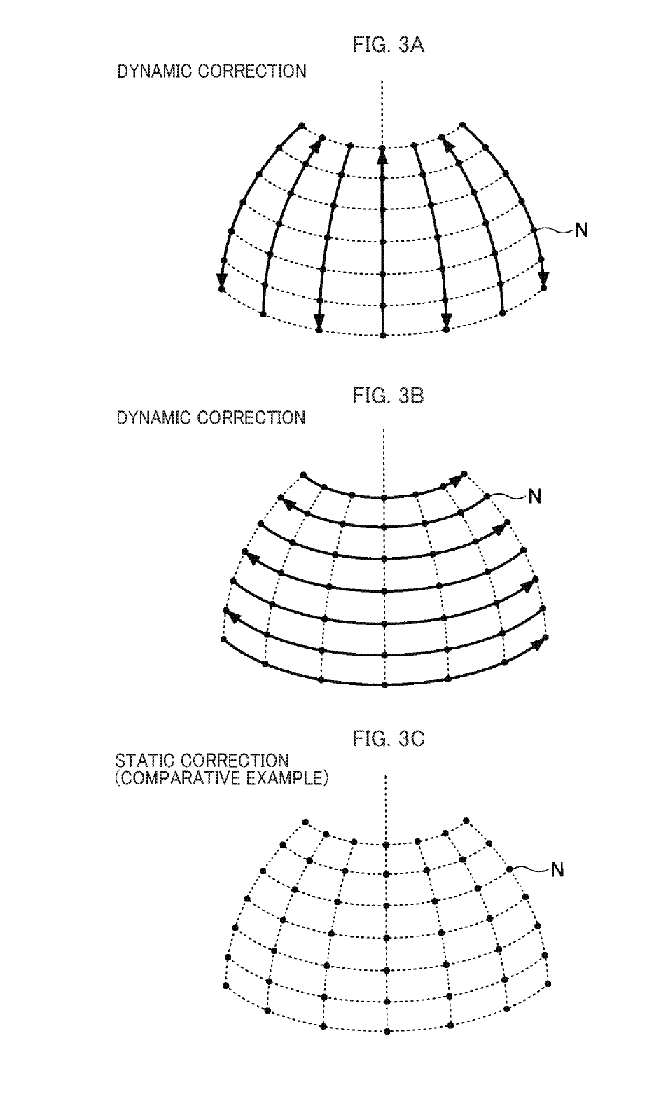

FIGS. 3A and 3B illustrate examples of dynamic position correction. FIG. 3A illustrates an example in which position correction is performed at each node N while the X-ray head 100 is being moved alternately in directions from top to bottom and bottom to top in the paper on which the figure is drawn, along longitudinal lines for movement on the virtual sphere S. When the X-ray head 100 is moved in a direction from the North Pole toward the equator on the virtual sphere S, a downward position deviation increases under the influence of gravity. When the X-ray head 100 is moved in a direction from the equator side toward the North Pole, there may be a slight downward deviation from a predetermined node. Thus, a correction amount when moving in the direction from the North Pole toward the equator on the virtual sphere S is set greater than a correction amount when moving in the direction from the equator toward the North Pole.

FIG. 3B illustrates an example in which position correction is performed at each node N while the X-ray head 100 is being moved alternately in directions from left to right and from right to left in the paper on which the figure is drawn, along latitudinal lines on the virtual sphere S. When the X-ray head 100 is moved in parallel to the latitudinal lines as well, flexure of the arm 210 and the X-ray head 100 varies, depending on the directions of movements and/or latitudes on the virtual sphere S, so that the amount of position deviation varies depending on the nodes. Thus, a correction amount at each of the nodes is determined according to the direction of movement and/or latitude. As one example, four correction values, in directions from top to bottom, from bottom to top, from left to right, and from right to left, may be set for one node.

FIG. 3C illustrates conventional static correction for comparison. The X-ray head 100 is stopped at each node and position correction is performed. Thus, it is not required to consider the influence of flexure which is exerted in different positions and states during movement, however, a treatment time increases as the number of nodes increases.

The dynamic corrections in FIGS. 3A and 3B may be performed for each patient in advance. However, alternatively, with a standard route used for treatment for the same affected area having been set, an amount of position correction at each of the nodes on the standard route may be determined in advance.

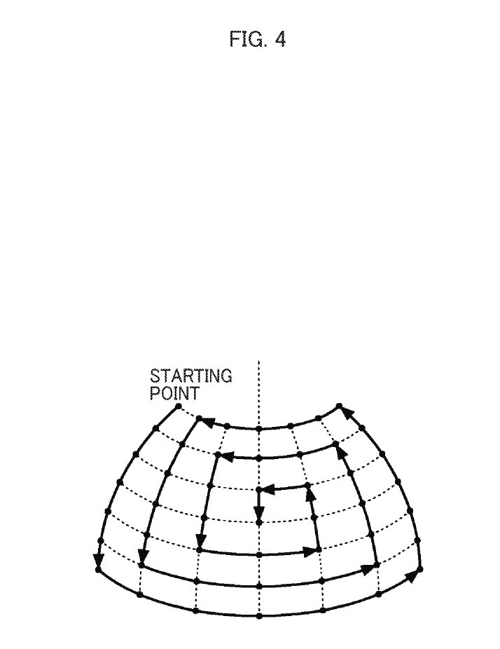

FIG. 4 illustrates an example of dynamic correction using a standard route. An optimal radiation route running through predetermined nodes is set in advance as a standard route, and a position correction value at each of the node is acquired while the X-ray head 100 is being continuously moved along the route. The optimal radiation route indicates, for example, a route having fewer changes in direction and more straight part and capable of being drawn with a continuous single stroke. In the dynamic position correction, a direction of movement serves as one reference, and a route capable of having more nodes in the same direction is desirable. As in FIG. 4, with provision of a radiation route running from the outside toward the inside on a rectangular spiral, the radiation head 100 can be continuously moved at a constant speed.

The standard route in FIG. 4 includes a path from the North Pole toward the equator, a path from the equator toward the North Pole, a path from left to right, and a path from right to left, and an individual position correction amount is set for each of the nodes.

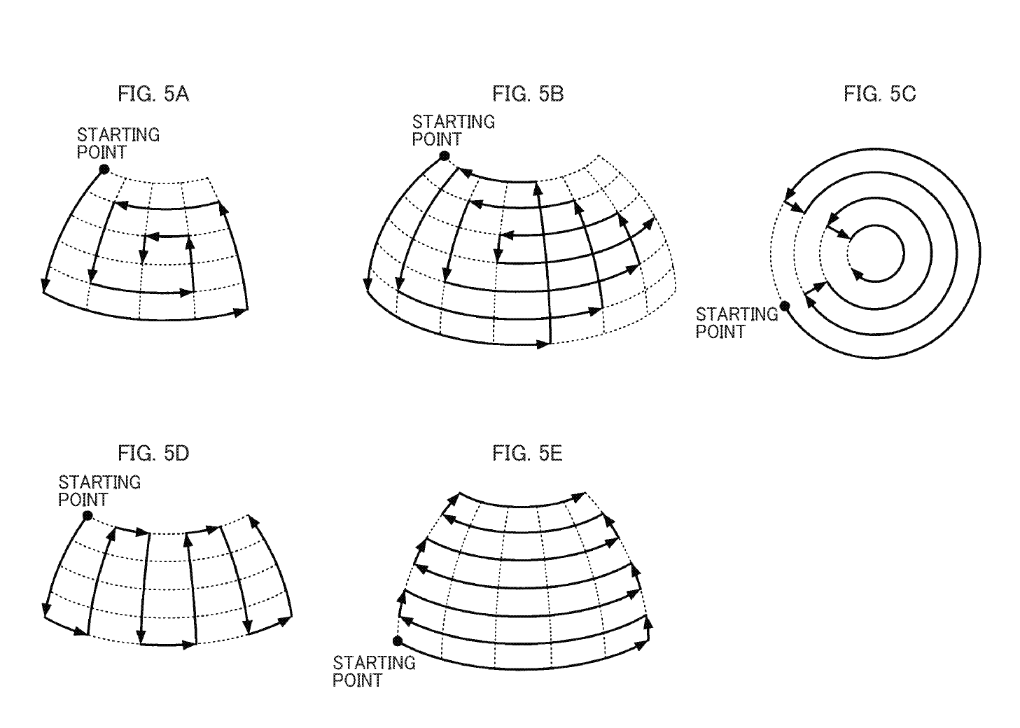

FIGS. 5A to 5E illustrate an examples of various standard routes. FIGS. 5A, 5B, 5D, and 5E are example patterns in which X-ray radiation is performed from a region at mid-latitude and low latitude, and FIG. 5C is an example pattern in which radiation is performed from a polar region. One or more standard routes are set for each treatment region, and a standard route is selected for treatment or each patient, or alternatively the standard routes may be combined. Such treatment regions may be classified as a head, a torso, etc., or classified according to the organ where a tumor appears. For each standard route, respective correction amounts at the nodes on the route are acquired and stored in advance, thereby being able to enhance the efficiency of treatment.

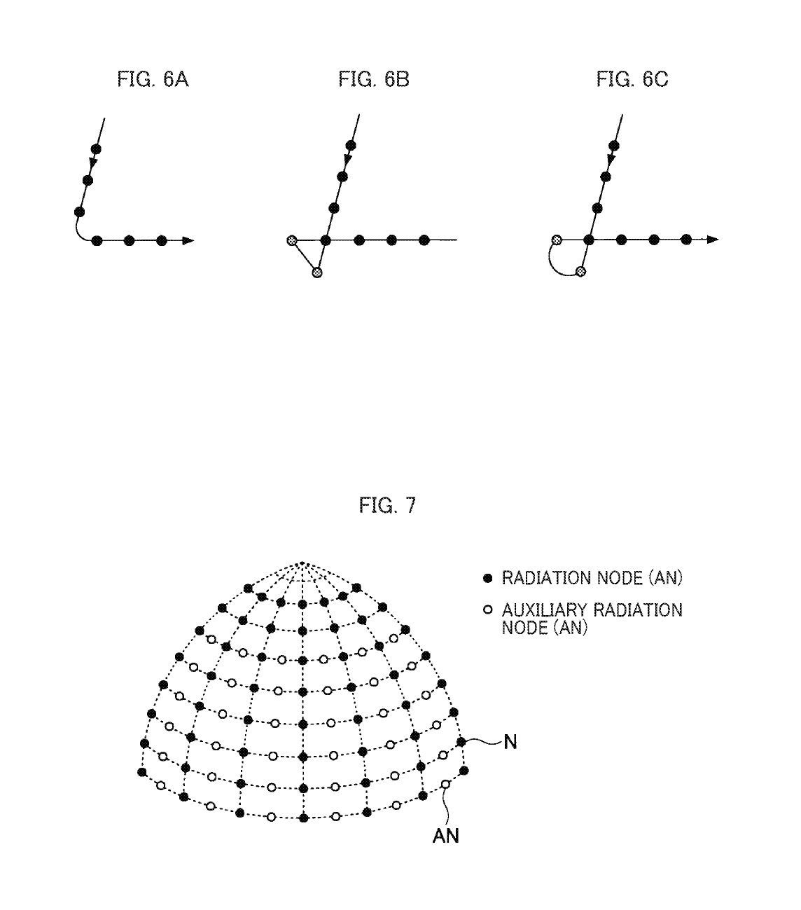

FIGS. 6A to 6C illustrate methods of changing direction of the X-ray head 100 during continuous movement. FIG. 6A is an example in which direction is changed with a predetermined radius of curvature. In this example, a node located at a corner cannot be used, however, since an abrupt change in direction is not included, the degree of reduction in speed is smaller and vibrations can be minimized. The excessively small radius of curvature may create an unusable node in the vicinity of a corner since vibrations are not stabilized. The excessively large radius of curvature may cause unnecessary travel time. It is desirable to provide a gentle curve having the relatively small radius of curvature which generates less centrifugal force.

FIG. 6B illustrates change in direction in a triangular type. In this method, a node located at a corner is usable. The total distance of movement of the X-ray head 100 is longer than the distance in FIG. 6A, however, nodes usable for treatment increase. Grey dots outside the corner are nodes for changing direction, at which radiation is not performed.

FIG. 6C illustrates a change in direction in a loop type. Since there is no edge as compared with FIG. 6B, direction can be changed with less vibration.

FIG. 7 is a diagram illustrating setting of auxiliary nodes. As described above, node-to-node distances vary depending on regions on the virtual sphere S having the radius R about the isocenter, even though having the same solid angle. A node-to-node distance at low latitude is longer than a node-to-node distance at high latitude. In the multidirectional radiation by a robot manipulator of the present embodiment, auxiliary radiation nodes (AN) are set between nodes in regions at low and middle latitudes, since it is desirable to perform radiation evenly at regular intervals of distance.

For example, assuming that a basic angle for setting radiation nodes is 10 degrees, each point of intersection of a longitudinal line and a latitudinal line at every 10 degrees is a radiation node (N). In this case, fewer nodes reduce correction time during movement, while increasing the number of auxiliary radiation nodes needed. A basic angle, for example, of 6 degrees or smaller and 3 degrees or greater is desirable. With a basic angle smaller than 3 degrees, a longer correction time during movement is required, and radiation fields may overlap at high latitudes. In a case of a basic angle of 5 degrees, for example, the auxiliary radiation nodes are not required at high latitude.

In FIG. 7, one auxiliary node is disposed between nodes immediately adjacent to each other in regions at low and middle latitudes, but the number of auxiliary radiation nodes may be increased near the equator. With the use of the auxiliary radiation nodes, regular intervals can be provided between radiation emission points on the virtual sphere S, as well as the radiation emission points can be prevented from overlapping.

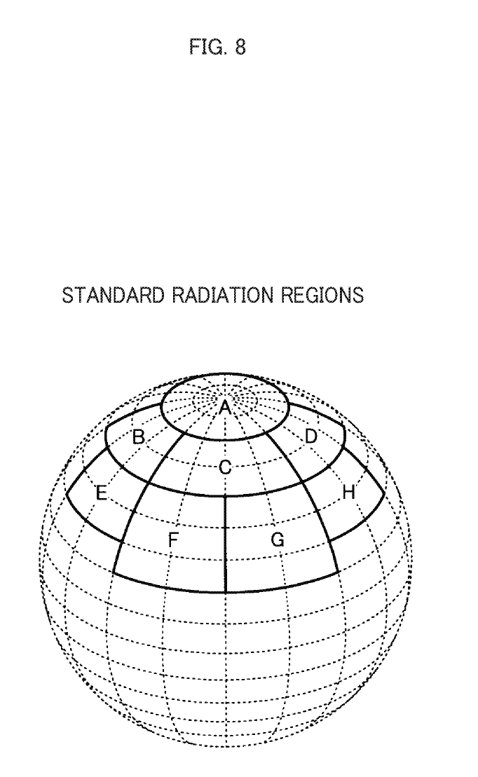

FIG. 8 is a diagram illustrating an example of setting of standard radiation regions (A to H) on the virtual sphere S. One or more standard routes may be set in each of the radiation region. Alternatively, a plurality of radiation regions may be combined to be used. A plurality of standard routes is set in each of the standard radiation regions, and a correction amount at each node corresponding to the direction and the position of the node may be measured and stored in advance for each standard route in each region. In this case, it is possible to select one or more radiation regions and radiation routes suitable for a patient among the plurality of standard radiation regions and routes.

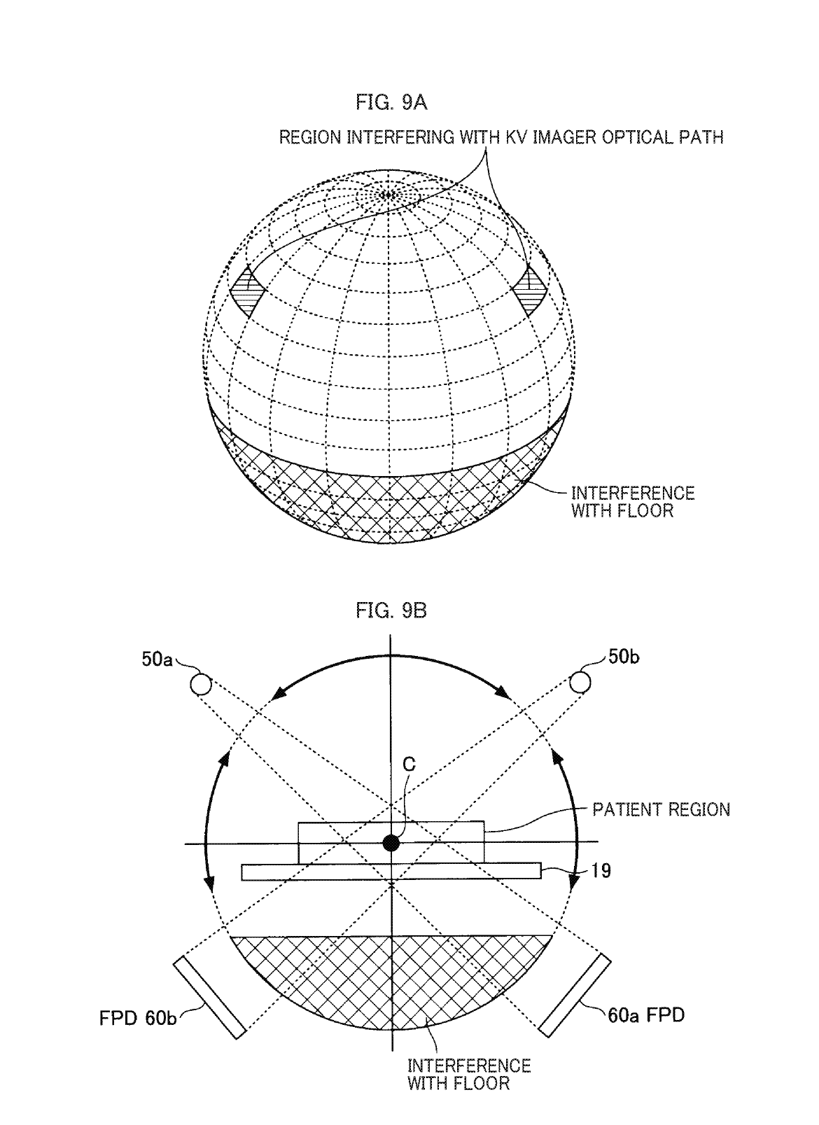

FIGS. 9A and 9B are diagrams illustrating setting of the radiation regions while interference is avoided. In creating a standard route, if there exists interference between an X-ray radiation optical path, a patient, the couch 19, a floor, or a kV imager optical path (an optical path determined by the positions of the X-ray tubes 50a, 50b and the FPDs 60a, 60b), etc., radiation may be affected. It is therefore desirable to create a radiation route eliminating such interference under conditions learned beforehand. For example, regions that have physical contact with the six-axis manipulator 200 including the X-ray head 100, the patient, the floor, the couch 19, etc., should be avoided Depending on the particular treatment room, walls and ceilings may have inadequate radiation shielding. In that case, X-ray radiation from below may be removed from a standard route as a constraint condition.

Ideally, the optical path of the kV imager should be avoided when setting the standard radiation route. However, since it is only a matter of inability of sensing caused by interference, in some cases a part corresponding to the kV imager path should be included in the standard route as a part to be passed through without radiation, rather than forcing creation of a route avoiding interference. From a view point of reduction in treatment time and securing of appropriate radiation emission points, a part in which radiation is not performed may be included in the route.

As one example, in the case of treatment of the head of a patient, several patterns of radiation routes can be set in regions where sensing of the kV imager is avoided, using a half (on the head side) of the virtual sphere S having a SAD of 600 mm. In the case of treatment of the torso of a patient, seven to ten patterns of radiation regions may be prepared where the SAD is set at 800 mm avoiding the optical path of the kV imager, a couch, a patient him/herself. Two to four patterns are selected and used among the radiation regions prepared for each treatment region, thereby being able to improve radiation speed and accuracy. The patterns of radiation regions may be formed incorporating safety mechanisms.

For example, an irradiation range with X-rays in the kV imager for detecting the gold marker should be narrowed as much as possible, however, since the position and the degree of motion of a tumor of a patient vary, optimization for each patient is difficult. When a standard route is created, a node at which radiation is not performed on the standard route may be provided on an individual patient basis, based on the premise that the kV imager is in a standard state. Alternatively, when a treatment plan is created, a standard radiation region not interfering with the radiation field of the kV imager may be selected depending on the patient.

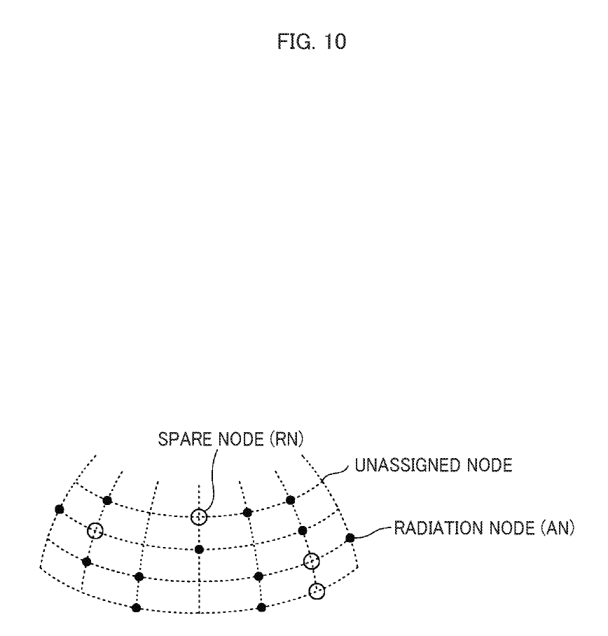

FIG. 10 is a diagram illustrating setting of the spare nodes. When actual radiation is performed, not all the nodes and auxiliary nodes on the selected standard route are used. To avoid a critical area, or to reduce irradiation of healthy cells by reducing overlap of X-ray radiation fields, only the nodes and auxiliary nodes scheduled under the treatment plan are to be used for radiation. In an embodiment, usable nodes are set as the spare nodes in case of occurrence of radiation errors.

The standard route includes many nodes (including radiation nodes and auxiliary nodes) to provide versatility. When nodes that are actually used for treatment are specified among the nodes on the standard route, nodes that are not used are set as the spare nodes in a treatment plan. The spare nodes thus set can be used for recovery from an event when radiation cannot be performed. For example, if the control device 120 (see FIGS. 2A and 2B) cannot acquire information on swinging movement of the collimator device within the X-ray head 100 due to sensing error of the kV imager or data transfer error, then dynamic tracking radiation cannot be performed. In this case, it is preferable to skip radiation at the scheduled node and perform radiation at the next spare node, rather than stop radiation altogether, so that treatment can be continued.

In the treatment plan, one or more patterns of the standard routes are selected from among the standard routes that are prepared for each treatment region, and when the nodes actually used on the standard route are determined, the spare nodes are set, in addition to the radiation nodes (including the auxiliary nodes depending on cases). For example, in the case where 200 nodes combining the radiation nodes and the auxiliary nodes are required for treatment, 240 nodes are set including the spare nodes. When two standard route patterns are combined to be used, 20 spare nodes are prepared for each pattern. At least two spare nodes are assigned on a straight part in each of the standard route patterns. Accordingly, even if a node at which radiation cannot be performed is generated during treatment, radiation can be performed at the spare node disposed next to the node.

Further, there are cases where treatment is restarted in the middle of the standard route, when a patient moves or treatment is temporarily interrupted due to a cough. When radiation performed at the radiation node has not reached the predetermined dose when interrupted, radiation should be restarted from the dose that is short at the previous radiation. However, radiation can be performed, at a dose corresponding to a shortage of the previous radiation, at the spare node located close to the interruption node along the radiation route, so that treatment can smoothly proceed.

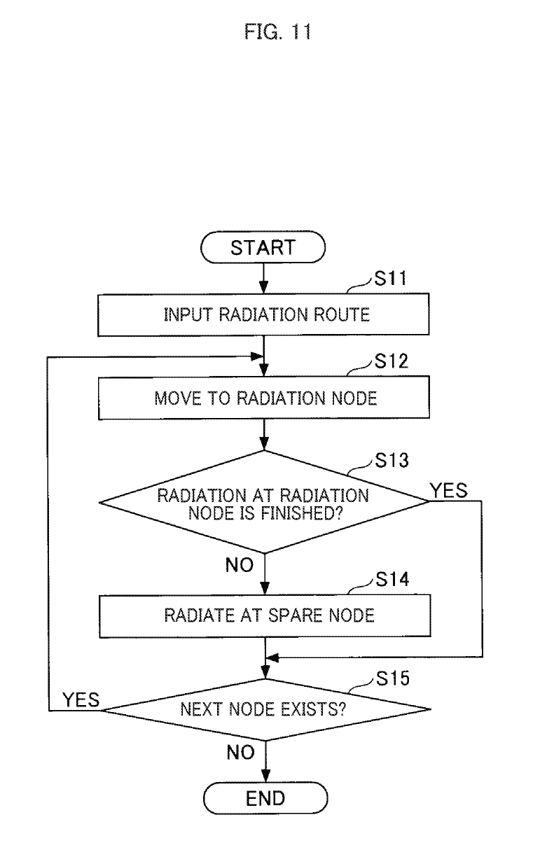

FIG. 11 is a flow chart illustrating an example of an operation of the radiation therapy apparatus 1 using the spare nodes. First, the radiation route used for treatment is inputted (S11). Along the radiation route, the spare nodes are set together with the radiation nodes and the auxiliary radiation nodes used for treatment. The six-axis manipulator 200 moves the X-ray head 100 to the radiation node along the radiation route (S12). Immediately after starting the treatment, the X-ray head 100 is moved to the first radiation node along the radiation route. The control device 120 controls radiation timing of the X-ray head 100 so as to issue a radiation instruction to the X-ray head 100 when the X-ray head 100 passes through the radiation node. The control device 120 determines whether radiation is finished at the scheduled radiation node (S13). When radiation is not finished at the scheduled radiation node (NO at S13), the control device 120 issues a radiation instruction to the X-ray head 100 when the X-ray head 100 passes through the closest spare node, and radiation is performed at the spare node (S14). When radiation at the radiation node is finished, the process proceeds to the next step S15. The six-axis manipulator 200 determines whether the next radiation node exists along the radiation route, and the processes of the step S12 and thereafter are repeated up to the last node (S15).

By this method, even if radiation is interrupted, radiation is continued at the spare node, thereby allowing treatment to continue.

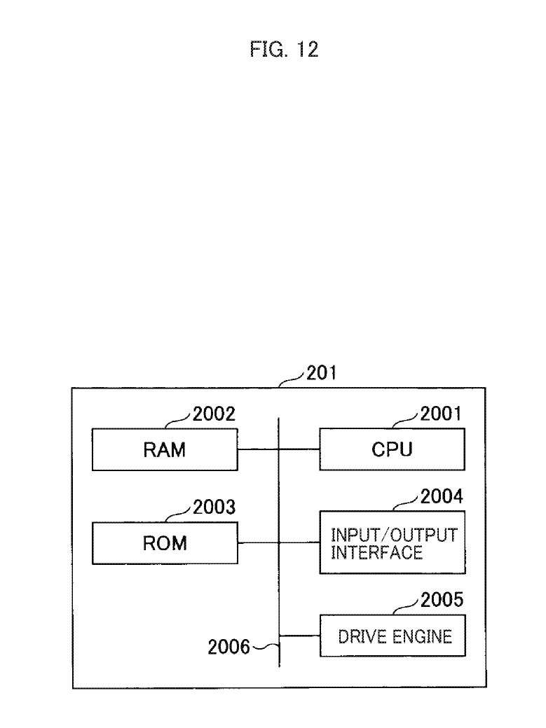

FIG. 12 is a hardware configuration diagram illustrating the manipulator sub-unit controller 201. The manipulator sub-unit controller 201 comprises a Central Processing Unit (CPU: arithmetic device) 2001, a Random Access Memory (RAM) 2002, a Read Only Memory (ROM) 2003, an input/output interface 2004, and a drive engine 2005, and they are connected through a bus 2006. The CPU 2001, the RAM 2002, and the ROM 2003 may be integrated on a single control substrate.

The CPU 2001 controls the entire operation of the six-axis manipulator 200. The RAM 2002 is used as a work area for the CPU 2001, and temporarily holds data used for position correction processing. The ROM 2003 holds route information that is inputted from the control device 120. The route information includes the standard route and speed information corresponding thereto. The ROM 2003 holds also information on the standard radiation regions on the virtual sphere S that are set in advance, and correction data for each node. When position correction for the X-ray head 100 is performed using a position correction program, the position correction program may be stored in the ROM 2003. The input/output interface 2004 includes a data input/output device that uses a predetermined communication protocol. The drive engine 2005 drives the X-ray head 100 with six axes (with six degrees of freedom).

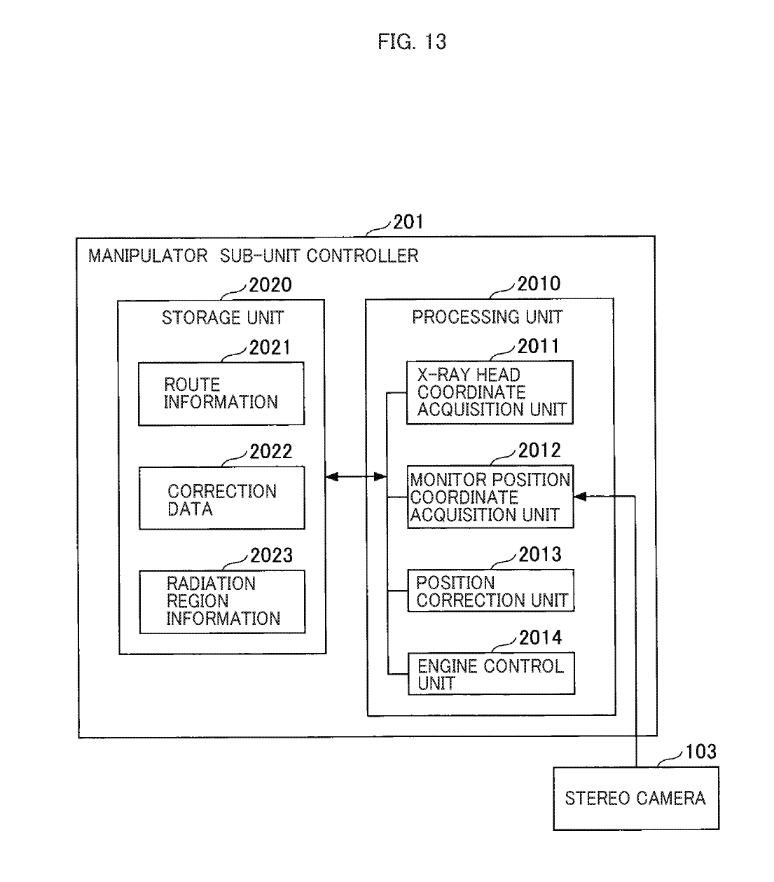

FIG. 13 is a functional block diagram illustrating the manipulator sub-unit controller 201. The manipulator sub-unit controller 201 comprises a processing unit 2010 and a storage unit 2020. The processing unit 2010 corresponds to the CPU 2001 of FIG. 12, and the storage unit 2020 corresponds to the ROM 2003 and RAM 2002.

The processing unit 2010 comprises an X-ray head coordinate acquisition unit 2011, a monitor position coordinate acquisition unit 2012, and a position correction unit 2013, and an engine control unit 2014. The storage unit 2020 stores route information 2021, correction data 2022, and radiation region information 2023. The route information 2021 includes radiation route(s) used for treatment and speed information on the X-ray head 100 corresponding to the radiation route(s). The radiation route(s) includes selected one or more standard routes. On the standard routes, the nodes actually used for treatment and the spare nodes may be set. The speed information may be a constant speed or speeds corresponding to parts, such as straight part, curved part, and corner part, constituting the radiation route.

The engine control unit 2014 controls drive of the six-axis manipulator 200 based on the route information 2021, the correction data 2022, and the radiation region information 2023. The X-ray head coordinate acquisition unit 2011 acquires coordinate position of the X-ray head 100 in a reference coordinate system where the isocenter is the origin, based on the positional coordinates, i.e., a drive amount, of the six-axis manipulator 200. The monitor position coordinate acquisition unit 2012 is used for dynamic position correction in advance, and acquires position deviation coordinates of the actual X-ray head 100 during continuous movement, based on image information from the stereo radiation axis monitoring camera 103. The position correction unit 2013 corrects the positional coordinates of the X-ray head 100 such that the small sphere 104a of the correction tool 104 is always positioned at the isocenter, that is, the X-ray head 100 passes through the nodes on a virtual sphere S determined by the SAD. The obtained correction value is stored in the storage unit 2020 as the correction data 2022.

Position correction at each node may be performed using the route information 2021 and the correction data 2022 when the standard route is used. For example, the selected route information 2021 is read, and a correction value corresponding to the direction of movement of the X-ray head 100 is selected from the correction data 2022, for each of the radiation nodes (including the auxiliary radiation nodes). The selected correction value may be stored in the storage unit 2020 as the correction data 2022 in association with the radiation route.

With this configuration, it is possible to enhance positional accuracy and radiation accuracy in performing radiation at each node while the X-ray head 100 is being moved on the predetermined radiation route.

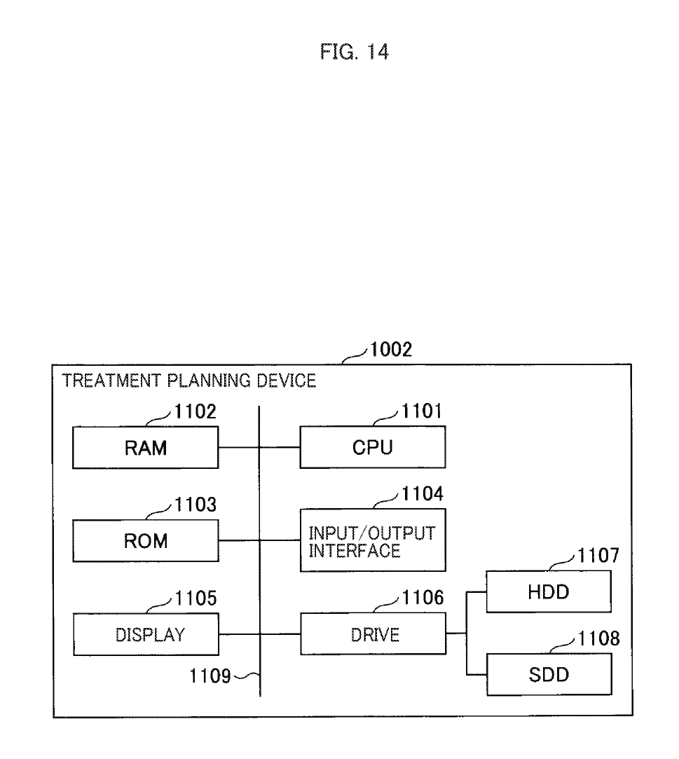

FIG. 14 is a diagram illustrating a hardware configuration of the treatment planning device 1002. The treatment planning device 1002 comprises a CPU 1101, a RAM 1102, a ROM 1103, an input/output interface 1104, a display 1105, a drive 1106, and a Hard Disk Drive (HDD) 1107 and/or a Solid State Drive (SDD) 1108, and they are connected through a bus 1109. The CPU 1101 controls the entire operation of the treatment planning device 1002. The RAM 1102 is used as a work area for the CPU 1101, temporarily holds data used for creation of a treatment plan including creation of a radiation route. The ROM 1103 stores information required for creation of a treatment plan. The input/output interface 1104 includes a data input/output device that uses a predetermined communication protocol. The drive 1106 reads/writes data with respect to the HDD 1107 and the SDD 1108 which are auxiliary storage devices. Further, the drive 1106 may reads/writes data with respect to an external storage device such as a removable disk. The display 1105 displays information required for an image, etc. When a treatment plan is created based on a treatment planning program, the treatment planning program is stored in the ROM 1103, the HDD 1107, or the SDD 1108.

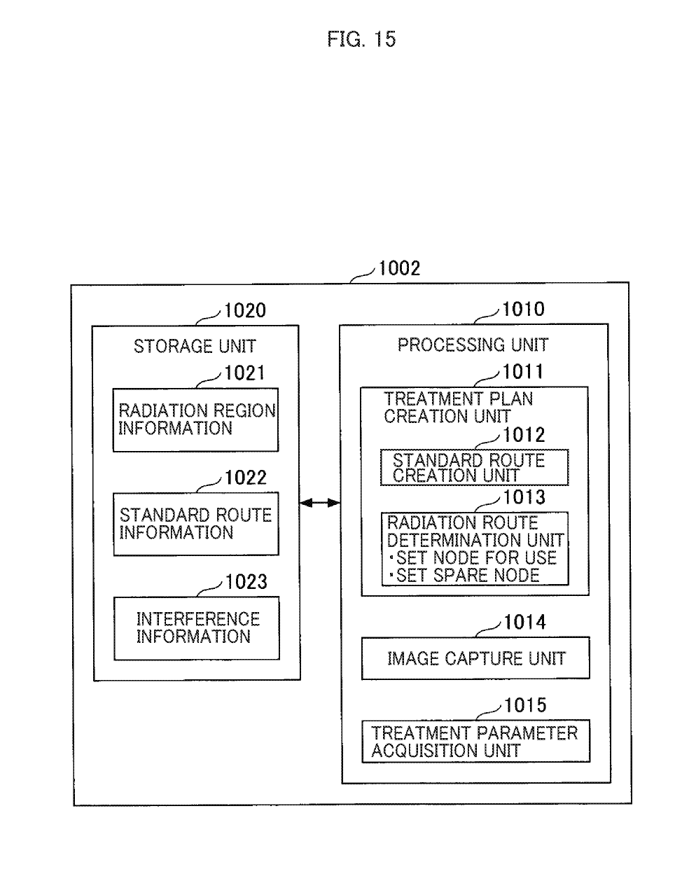

FIG. 15 is a functional block diagram illustrating the treatment planning device 1002. The treatment planning device 1002 comprises a processing unit 1010 and a storage unit 1020. The processing unit 1010 corresponds to the CPU 1101 of FIG. 14, and the storage unit 1020 corresponds to at least a part of the ROM 1103, the HDD 1107, and the SDD 1108.

The storage unit 1020 stores radiation region information 1021, standard route information 1022, and interference information 1023. The radiation region information 1021 includes a standard radiation region of FIG. 8. The standard route information 1022 includes at least one optimal radiation route pattern for each affected area. The affected area may be broadly classified according to body parts (head, torso, abdomen, respiratory organs, circulatory system, extremities, etc.), or according to organs. The standard route information 1022 may be stored in association with radiation regions. In this case, one or more standard routes are associated with each of the radiation regions. The interference information 1023 includes information on the floor, the optical path of the kV imager, the couch, a body shape of a patient, etc.

The processing unit 1010 comprises a treatment plan creation unit 1011, an image capture unit 1014, and a treatment parameter acquisition unit 1015. The treatment plan creation unit 1011 includes a standard route creation unit 1012, and a radiation route determination unit 1013. The standard route creation unit 1012 formulates a standard route that is an optimal movement route of the X-ray head for each affected area, each patient, and each standard radiation region. The term “optimal movement route” means a route on which radiation can be performed at corrected positional coordinates without stopping the X-ray head 100 at each radiation node, and preferably, a route on which variation in speed of the X-ray head 100 is minimized. For example, such a route is desirable that has more straight part and less change in direction, and if possible, can be drawn with a continuous single stroke. Further, it is preferable to create a route that avoids interference with reference to the interference information 1023. Depending on the radiation regions, the auxiliary radiation node(s) may be included in the standard route. The formulated standard route is stored in the storage unit 1020 as the standard route information 1022.

The image capture unit 1014 acquires a CT image of an affected area and an image on a body surface captured by the body surface monitoring camera 102. The treatment parameter acquisition unit 1015 receives inputs of treatment parameters by a physician. The treatment parameters include a treatment region, a critical area, a collimator radiation field, the number of radiation nodes, and a concentrated radiation dosage target, etc.

The radiation route determination unit 1013 selects one or more routes for each patient from the standard routes based on the inputted treatment parameters, and sets a node for use that is actually used for radiation along the radiation route, and a spare node. The determined radiation route may be stored in the storage unit 1020.

In an embodiment, in addition to continuous movement radiation of the X-ray head 100, dynamic tracking radiation is performed at each node. Thus, swinging collimating of the X-ray head 100 will be described hereinafter.

### Configuration of the X-ray Head 100

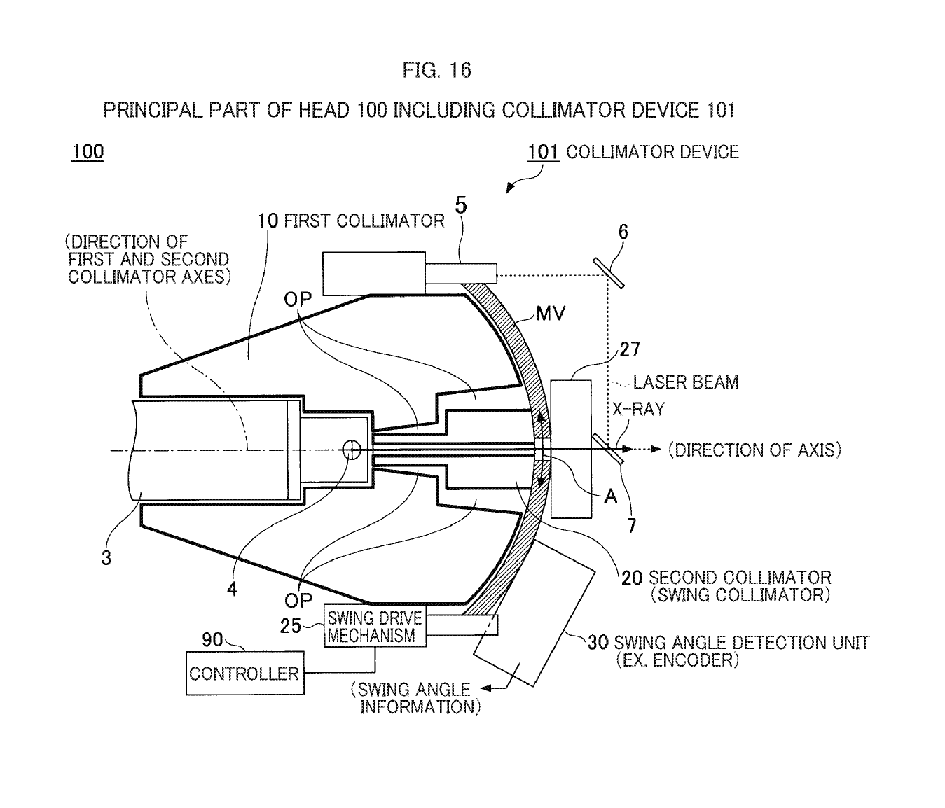

FIG. 16 is a schematic view illustrating a principal part of the X-ray head 100. The X-ray head 100 functions as a radiation apparatus, and comprises a collimator device 101 in its principal part. The collimator device 101 comprises a target 4 for generating X-rays, a first collimator 10, a second collimator 20, and a swing drive mechanism 25 to swing the second collimator 20 within the first collimator 10. An actual radiation field is formed by the first collimator 10, the second collimator 20, and a swing drive mechanism 25 operating together.

The first collimator 10 has a center axis in a direction indicated by a dot-and-dash line, and has a shape that is symmetrical about the center axis. The center axes of an acceleration tube 3 and the target 4 are aligned with the axis of the first collimator 10 such that the axis direction of the first collimator 10 is aligned with the traveling direction of accelerating electron beams.

The second collimator 20 is disposed, with a gap (OP), within the first collimator, and allows X-rays generated in the target 4 to pass therethrough in the axis direction of itself. In the drawing, a line of an arrow illustrated with a thick line in the lateral direction from the target 4 indicates the direction of the X-ray radiation. The first collimator 10, the second collimator 20, and the target 4 are made of, for example, tungsten (W). A dosimeter for measuring a dose of X-rays, e.g., an ion chamber 27, is provided on the emitting side of the second collimator 20.

A laser targeting unit 5 is provided, which is configured to emit visible laser light (e.g., red light) onto a surface of a suitable member coupled to the first collimator 10. The optical axis of the visible laser light emitted from the laser targeting unit 5 is aligned with the X-ray axis along the traveling direction of the X-rays using an optical system such as mirrors 6 and 7. The X-ray-incident position can be found by observing the body surface of the patient painted with the visible laser light.

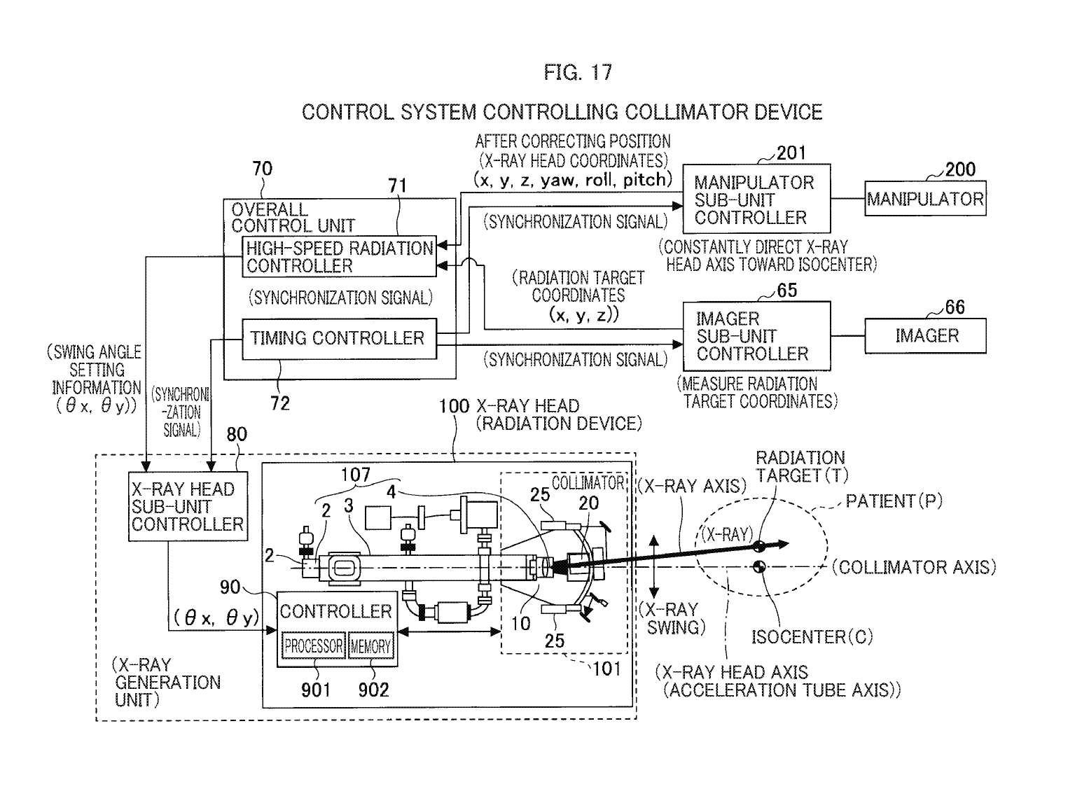

The swing drive mechanism 25 is configured to move a movable member MV based on a control signal from a controller 90 (see FIG. 17) installed in the X-ray head 100, so that the second collimator 20 connected to the movable member MV is rotated (swung) in the two directions indicated by arrow A.

The target 4 is positioned on the axis of the second collimator 20. For example, bearings are provided between the virtual sphere of the first collimator, which is a surface of the sphere whose center is the target 4, and the movable member MV coupled to the second collimator 20. The bearings are coupling members (linear slides) comprising arc-shaped curvilinear-motion bearings in two directions so as to allow movements of two-degree-of-freedom, for example. The insertion of the bearings supports the movable member MV as well as enables smooth swinging movement of the second collimator 20 while the target 4 serves as the rotational center. A portion relating to the swinging movement, which includes the second collimator, the bearings, and the movable member MV, is referred to as the “swing portion”. The swing drive mechanism 25 is configured to swing the swing portion to the desired position, so that the affected area can be accurately irradiated at each of the radiation nodes.

A swing angle detection unit 30 is provided as an example of a deviation detection means. The swing angle detection unit 30 is configured to detect swing displacement (swing angle) relative to the reference point of the second collimator 20 to output its result as swing angle information (or displacement information). The swing angle detection unit 30 may be an optical angle measurement device of an autocollimator type which uses a semiconductor laser and optical system, or an angle detector of an encoder type which uses an encoder sensor.

Electron beams emitted from an electron gun 2 (see FIGS. 17), installed in the X-ray head 100, are accelerated through the acceleration tube 3 to collide with the target 4, so that the electron beams are converted into X-rays. Thus, the target 4 is an X-ray generation source. The radiation area of X-rays generated by the target 4 is narrowed by the second collimator 20, to form a predetermined radiation field for an affected area. The first collimator 10 restrains the X-rays generated by the target 4 from leaking to the exterior.

Under the drive control of the swing drive mechanism 25, the movable member MV is moved in the direction indicated by the arrow A (up and down direction in the drawing), so that the second collimator 20 swings in the direction of the arrow A. The swing displacement (swing angle), which is a swing amount relative to the reference point, is detected by the swing angle detection unit 30. The detected swing displacement is used, for example, for feedback control and feedforward control, to control the swinging movement, so that the movement is stabilized. The movable member MV is capable of swinging not only in the direction of the arrow A (one-dimensional operation), but also the direction vertical to the paper on which the figure is drawn (front and back direction). Accordingly, the second collimator 20 is capable of swinging in the front and back direction of the paper, and thus the second collimator 20 is configured to swing in two directions (two dimensionally) of the up and down direction and the vertical direction of the paper. Such two-dimensional swing drive may be performed using the swing drive mechanisms 25 individually provided for the directions. Further, detection/output of swing displacement can be performed by the single swing angle detection unit 30, or the swing angle detection units 30 individually provided for the directions. The swing drive mechanism 25 is configured, for example, with a voice coil motor, thereby enabling swinging movement with higher speed and accuracy.

The X-ray head 100 further includes the electron gun 2 and the controller 90, which will be described later, in addition to the acceleration tube 3, the target 4, the laser targeting unit 5, the first collimator 10, the second collimator 20, the swing drive mechanism 25, and the swing angle detection unit 30 in FIG. 16.

### Collimator Drive Control System

FIG. 17 is a diagram illustrates the outline of a control system of the collimator device 101. The collimator device 101 is controlled using the overall control unit 70, the X-ray head sub-unit controller 80, and the controller 90 in the X-ray head 100, the manipulator sub-unit controller 201, and the imager sub-unit controller 65. The manipulator sub-unit controller 201 controls the six-axis manipulator 200 may be included in the overall control unit 70 or the control device 120 together with the overall control unit 70 as illustrated in FIG. 2A. The X-ray head sub-unit controller 80 may be included in the control device 120 or in the X-ray head 100. Alternatively, the sub-unit controller 80 may be disposed, as a microchip, outside the X-ray head 100.

The overall control unit 70 comprises a high-speed radiation controller 71 and a timing controller 72. The timing controller 72 is configured to generate synchronization signals for synchronizing the devices in the radiation therapy apparatus 1, and supply the synchronization signals to the manipulator sub-unit controller 201, the imager sub-unit controller 65, the X-ray head sub-unit controller 80, and the like. The imager sub-unit controller 65 is connected to the imager 66. The imager 66 is a kV imager that includes a set of the X-ray tube 50a and the FPD 60a and another set of the X-ray tube 50b and the FPD60b, described above, and captures an X-ray image in the vicinity of a tumor. The imager sub-unit controller 65 processes the acquired X-ray image to calculate radiation target coordinates.

The high-speed radiation controller 71 is configured to receive the coordinates (x, y, z, yaw, roll, pitch) of the X-ray head 100 from the manipulator sub-unit controller 201 that controls the six-axis manipulator 200. Under control of the manipulator sub-unit controller 201, the X-ray head 100 is moved along a predetermined radiation path on the virtual sphere S having an SAD so as to be directed toward the isocenter at all times. As a coordinate system, the isocenter C is set as the origin, the x-axis and the y-axis are set in two directions on a plane, and the z-axis is set in a direction vertical to the xy plane. The “yaw” is the amount of rotation about the Z-axis, the “roll” is the amount of rotation about the x-axis, and the “pitch” is the amount of rotation about the y-axis.

The high-speed radiation controller 71 is configured to receive also the coordinates (x, y, z) of the radiation target (a tumor, etc.,) from the imager sub-unit controller 65, and calculate swing angles θx and θy at which the second collimator 20 should be positioned, based on the coordinates (x, y, z) of the radiation target and the current coordinates (x, y, z, yaw, roll, pitch) of the X-ray head 100. The obtained swing angles are supplied to the X-ray head sub-unit controller 80 as swing angle setting information (θx, θy).

The X-ray head sub-unit controller 80 is configured to receive the swing angle setting information (θx, θy) from the high-speed radiation controller 71, and supply the swing angle setting information (θx, θy) to the controller 90 installed in the X-ray head 100. Such swing angle setting may be directly inputted from the high-speed radiation controller 71 to the controller 90 in the X-ray head 100.

The controller 90 in the X-ray head 100 is configured to receive the swing angle setting information, perform arithmetic processing for feedback and feedforward control, and output such control information to the swing drive mechanism 25 of the collimator device 101. The controller 90 may be a control IC including a processor 901 and memory 902. The swing drive mechanism 25 is configured to swing the swing portion including the second collimator based on the received control information, so that a swing angle results in the swing angle specified by the swing angle setting information (θx, θy). Accordingly, the X-ray head 100 radiates X-rays while performing dynamic tracking, when passing through each of the radiation nodes along the radiation route. In a case where the controller 90 cannot receive swing angle information from the overall control unit 70, dynamic tracking radiation may be performed at a spare node at the next timing when the swing angle information is received.

By repeating the above-described operations, the X-ray axis is directed toward the radiation target at all times, and even when a body is in motion, the affected area is appropriately irradiated with X-rays. As illustrated in the lower right side of FIG. 17, even if a radiation target T of the patient P deviates from the collimator axis serving as the reference axis of radiation, the X-ray axis tracks the target (see a thick arrow) with the swinging movement (see two-headed arrow) of the collimator. Such a sequence of operations is implemented on a real-time basis in accordance with the synchronization signals that are generated and outputted by the timing controller 72 in the overall control unit 70, thereby being able to perform dynamic tracking at a higher speed.

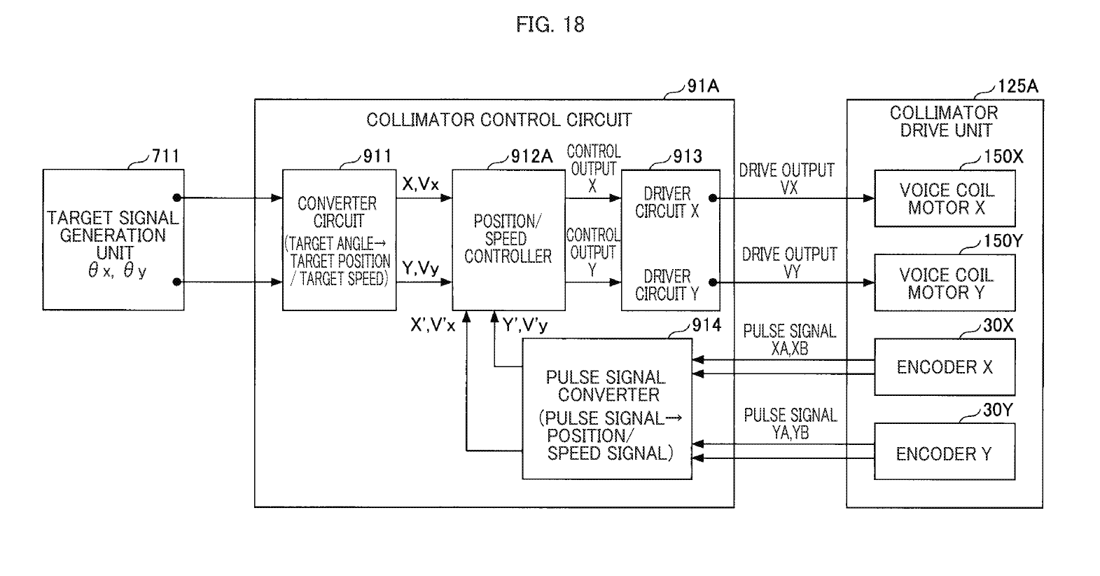

FIG. 18 is a block diagram illustrating a system configured to perform drive control of a collimator device. In an embodiment, swing control is performed while continuously moving the X-ray head 100, so that the X-rays are radiated while tracking body motion at each of the radiation nodes.

The drive control system in FIG. 18 includes a target signal generation unit 711, a collimator control circuit 91A, and a collimator drive unit 125A. The target signal generation unit 711 is implemented by the action of the high-speed radiation controller 71. The collimator control circuit 91A is included in the controller 90 in the X-ray head 100. The collimator drive unit 125A includes the swing drive mechanism 25 and the swing angle detection unit 30 of FIG. 16. In this example, voice coil motors 150X, 150Y are used as the drive mechanism 25, while encoders 30X, 30Y are used as the swing angle detection unit 30.

The collimator control circuit 91A comprises a converter circuit 911, a position/speed controller 912A, a driver circuit 913, and a pulse signal converter 914. Target signals θx, θy, indicative of the swing angle of the target, are inputted from the target signal generation unit 711 via the X-ray head sub-unit controller 80 to the converter circuit 911. The converter circuit 911 is configured to convert the inputted angle information into target position (X, Y) and target speed (Vx, Vy), and supply such conversion results to the position/speed controller 912A.

On the other hand, pulse signals XA, XB and YA, YB indicative of current angle information on the second collimator 20 are inputted from the encoders 30X, 30Y in the collimator drive unit 125A to the pulse signal converter 914. A pair of pulse signals XA, XB indicates a phase difference about the X-axis, while a pair of pulse signals YA, YB indicates a phase difference about the Y-axis. The pulse signal converter 914 is configured to transform the inputted pulse signals XA, XB and YA, YB into a detection position (X′, Y′) and a detection speed (V′x, V′y), and supply such transformation results to the position/speed controller 912A.

The position/speed controller 912A is configured to generate control signals X, Y for the position and speed of the second collimator 20 to track the moving body, based on the target position (X, Y), the target speed (Vx, Vy), the detection position (X′, Y′), and the detection speed (V′x, V′y), and output the signals to the driver circuit 913. The driver circuit 913 includes a driver circuit X configured to perform drive control of the voice coil motor 150X, and a driver circuit Y configured to perform drive control of the voice coil motor 150Y. The driver circuit X is configured to output a drive signal VX to a voice coil motor X. The driver circuit Y is configured to output a drive signal VY to a voice coil motor Y.

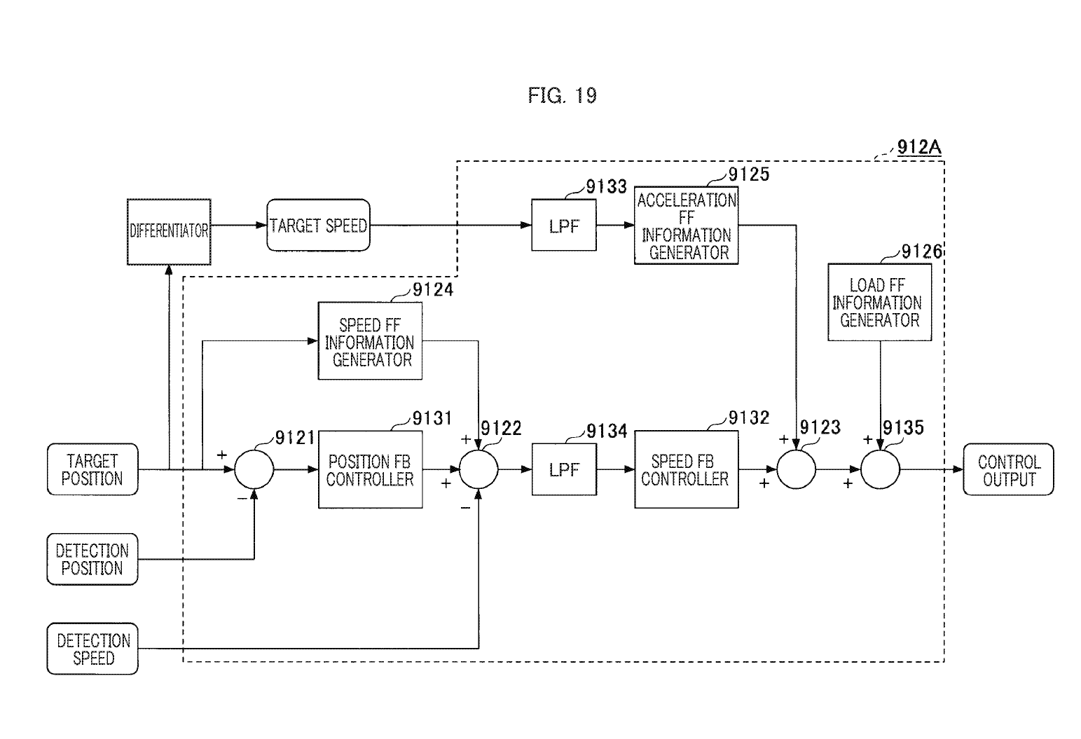

FIG. 19 illustrates generation of control outputs implemented in the position/speed controller 912A. It is difficult to deliver high-precision tracking performance using only feedback control, with respect to a target, such as a lung tumor, which moves in a complex manner involving both acceleration and deceleration. In order to solve such a problem, the position/speed controller 912A implements feedforward control with excellent tracking performance.

An X-ray radiation target position and a target speed obtained by differentiating the target position are inputted from the converter circuit 911. Further, the detection position and the detection speed indicative of the actual irradiation position and speed are inputted from the pulse signal converter 914.

The position/speed controller 912A comprises a first arithmetic circuit 9121, a second arithmetic circuit 9122, a third arithmetic circuit 9123, a fourth arithmetic circuit 9135, a speed FF information generator 9124, an acceleration FF information generator 9125, a load FF information generator 9126, a position FB controller 9131, a speed FB controller 9132, and low-pass filters 9133, 9134.

The first arithmetic circuit 9121 is configured to calculate a difference between the target position and the detection position, and output such arithmetic result to the position FB controller 9131. The position FB controller 9131 is configured to generate position feedback (FB) information.

The speed FF information generator 9124 is configured to differentiate the target position to generate speed FF information.

The second arithmetic circuit 9122 is configured to perform an arithmetic operation based on three pieces of information, which are position FB information outputted from the position FB controller 9131, speed FF information outputted from the speed FF information generator 9124, and the detection speed. More specifically, a difference between the speed FF information and the detection speed is obtained, and this difference and the position FB information are added together. The operation result is subjected to noise filtration and waveform shaping in the LPF 9134, and inputted to the speed FB controller 9132. The speed FB controller 9132 is configured to output speed FB information.

The acceleration FF information generator 9125 is configured to generate the acceleration FF information based on the target speed. The target speed is calculated by differentiating the target position, and a differential signal is subjected to noise filtration and waveform shaping in the LPF 9133, and then is inputted to the acceleration FF information generator 9125. The acceleration FF information is used for performing control so as to cancel acceleration torque generated by the moment of inertia (inertia) of mechanical parts during acceleration.

The third arithmetic circuit 9123 is configured to add the speed FB information outputted from the speed FB controller 9132 and the acceleration FF information outputted from the acceleration FF information generator 9125. The fourth arithmetic circuit 9135 is configured to generate a control signal based on an addition result outputted from the third arithmetic circuit 9123 and load FF information generated in the load FF information generator 9126, and output a control signal. Load FF information included in the control signal serves to cancel an expected mechanical load. Change in inertia can be restrained by the acceleration FF information contained in the control signal, and change in mechanical load can be handled with the load FF information.

An acceleration FF gain is defined by a reciprocal of a control target model including mechanical inertia. The speed FF is a differential gain. Proportional (P) control may be used for the position FB, while all or a part of Proportional-Integral-Differential (PID) control may be used for the speed FB, or a lead-lag filter may be used therefor.

Control in FIG. 19 may be performed through implementation of an algorithm, or may be performed using a digital circuit or an analog circuit integrated in an IC chip.

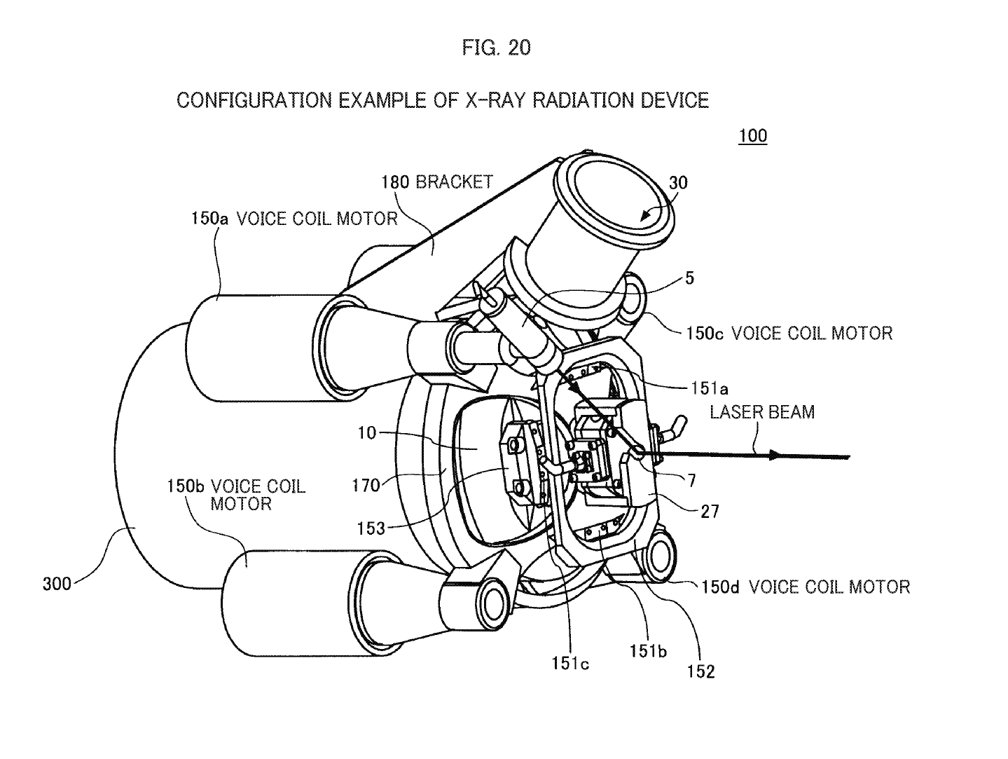

FIG. 20 is a perspective view illustrating a configuration example of the X-ray head 100. The X-ray head 100 functions as the X-ray radiation device, and is housed in an X-ray head base 300 serving as housing. The X-ray head base 300 has a hollow cylindrical shape, and houses the first collimator 10 in such a manner as to close an opening of its cylindrical body on one end side (X-ray emitting side). The target 4, which converts the electron beams emitted from the electron gun 2 (see FIG. 17) into X-rays, is provided on the axis of the first collimator 10 (see FIG. 16).

Four voice coil motors 150a, 150b, 150c, 150d are provided on an outer peripheral surface of the X-ray head base 300, and are configured to drive the swing portion including the second collimator 20 to swing at least in two directions orthogonal to each other. The voice coil motors 150a, 150b, 150c, 150d are each disposed every quarter of the way around a circumference of the cylindrical body (i.e., at intervals of 90 degrees). The swing angle detection unit 30 is fixed and connected to the end part of a bracket 180 extending from the outer peripheral surface of the X-ray head base 300. In this example, the swing angle detection unit 30 is an autocollimator using a semiconductor laser and an optical system, and a reflective mirror is fixed to the outer surface of a swing base 170 (corresponding to the movable member MV in FIG. 15) opposed to the swing angle detection unit 30. The reflective mirror is not illustrated in the perspective view of FIG. 19.

The end part (X-ray emitting side) of the voice coil motor 150a is provided with the laser targeting unit 5 via an appropriate member. The optical axis of the visible laser light emitted from the laser targeting unit 5 is aligned with the X-ray axis using the optical system configured with a mirror 7, etc. Thus, the irradiation target of the X-rays can be confirmed by visually checking the visible laser light. The ion chamber 27 is fixed, using an appropriate member, on the outer surface side of the swing base 170 between the mirror 7 and the second collimator 20 (see FIG. 16), and measures a radiation dose, the direction of irradiation, etc.

Arc-shaped curvilinear-motion bearings 151a and 151b are disposed between an intermediate member 152 and the swing base 170 coupled to the second collimator 20. Further, arc-shaped curvilinear-motion bearings 151c and 151d that are directed in a direction orthogonal to the direction of the curvilinear-motion bearings 151a, 151b are disposed between the intermediate member 152 and a mounting base 153 on the first collimator 10 (the curvilinear-motion bearing 151d is not illustrated in FIG. 16). With this configuration, the swing base 170 makes smooth swinging movements.

The rotational center of the swing portion including components, such as the second collimator 20, the swing base 170, and the curvilinear-motion bearings 151a to 151d, is substantially aligned with the center of gravity of the swing portion. The rotational center of the swing portion is positioned at the target 4 that generates X-rays. Deviation in the center of gravity of the swing portion causes application of loads varying with the directions of swing, whereas, by aligning the rotational center of the swing portion with the center of gravity, such gravity loads with respect to a coefficient of friction between a fixed portion and a movable portion can be brought close to zero. Accordingly, even if the posture of the X-ray head 100 changes with irradiation in multiple directions, it is possible to minimize change in mechanical load, such as variation in friction force affected by gravity.

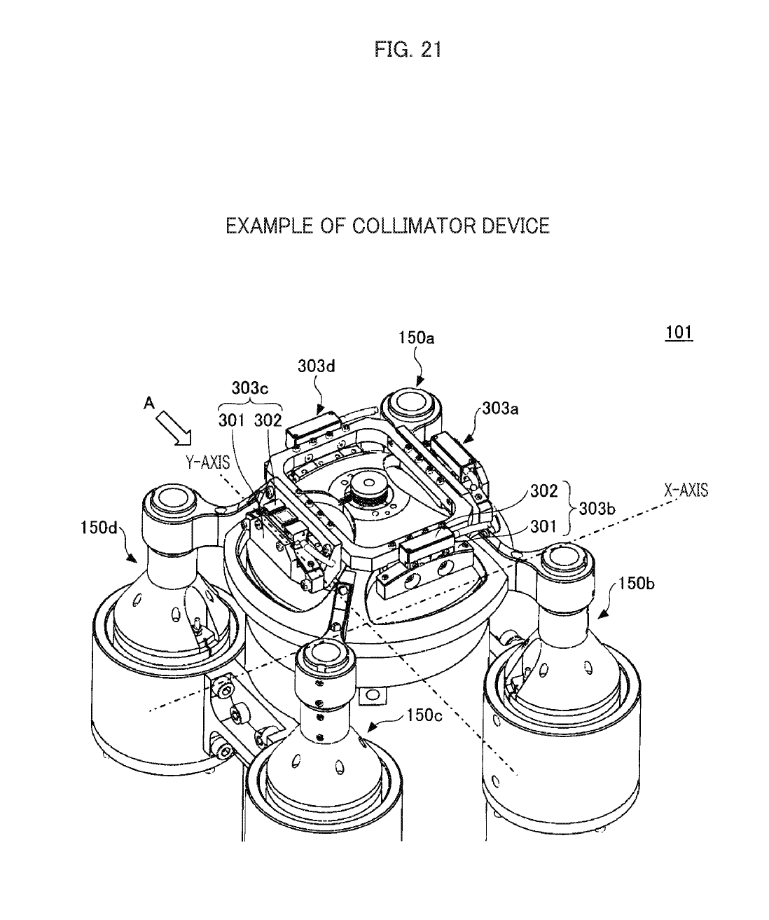

FIG. 21 is a perspective view illustrating another example of the collimator device 101. In this example, an encoder system is employed as a displacement detection means, i.e., the swing angle detection unit 30, instead of an autocollimator system. Two encoders 303b, 303d are disposed along the X-axis, while two encoders 303a, 303c are disposed along the Y-axis (collectively, referred to as “encoders 303”). The encoders 303 each include an encoder sensor 302 and a linear scale 301.

The encoders 303 include a magnetic encoder, which is excellent in environmental robustness against dust, oil, etc., and a low-cost optical reflective encoder, which can be used where a favorable environment permits. The encoder output types include an incremental type and an absolute type. The incremental type requires determining the origin every time when a power supply is turned on/off. The absolute type does not require such operation since position information is recorded.

The encoders 303 serving as a displacement detection sensor are important in performing drive control, and thus it is preferable to employ redundancy, that is, to dispose them in sets. Thus, two sets of encoders 303 are disposed to detect each of angles (θx, θy) about the X-axis and the Y-axis, thereby providing backup in the event of failure of the sensor or malfunction of the sensor itself. The same type of encoders may be used for two sets of encoders in each axis direction, while the magnetic encoders and the optical encoders may be used in combination.

In the configuration in FIG. 20, the voice coil motors 150a to 150d are disposed such that the drive axes thereof are tilted at 45 degrees with respect to the X-axis and Y-axis serving as references. In terms of layout, the drive axes of the voice coil motors are set at 45 degrees relative to the reference axes, so that the arrangement of the encoders 303a to 303d is facilitated and the collimator device 101 is downsized.

However, the drive axes of the voice coil motors 150a to 150d may be aligned with the X-axis and Y-axis which are the references. In this case, the drive axes of the voice coil motors 150a to 150d are parallel to the orientation of the encoders 303a to 303d, which simplifies position-to-angle conversion, resulting in enhanced control accuracy.

The point of intersection of the X-axis and Y-axis constituting reference coordinates is the location of the target 4, which is the X-ray source, and also the rotational center of the swinging movement of the swing portion configured with the second collimator 20 and components mounted thereto. As described above, the location of the target 4 serving as the rotational center is aligned with the center of gravity of the swing portion.

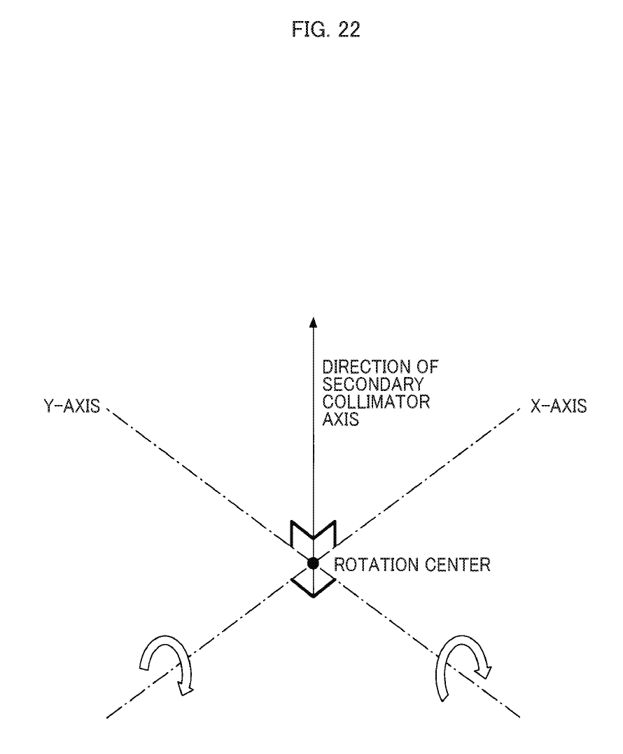

FIG. 22 is a diagram illustrating the X-axis and Y-axis serving as the rotation axes of the swinging movements of the second collimator 20. The point of intersection of the X-axis and Y-axis is the location of the target, which is the X-ray source, and the rotational center about which the second collimator 20 is swung in two directions orthogonal to each other. The moment of inertia of the components of the swing portion, configured with the second collimator and the components mounted thereto, is substantially equal about each of the X-axis and the Y-axis, which are the reference axes in two directions orthogonal to each other passing through the rotational center of the swinging movement. With such an arrangement, the effects caused by imbalance in inertia can be cancelled using the main parts of acceleration-FF-control parameters. As a result, tracking performance can be stabilized against acceleration and deceleration of the target (affected area).

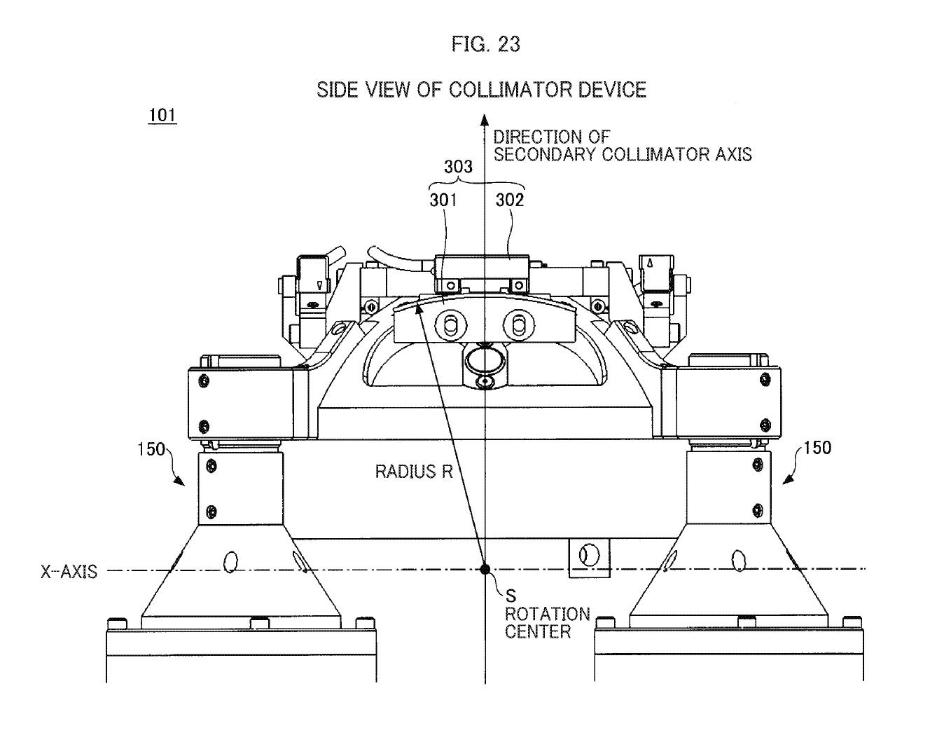

FIG. 23 is a side view illustrating the collimator device 101. The linear scale 301 of the encoder 303 is mounted as an arc of a curve having a radius R with respect to the X-axis and Y-axis passing through the center point of the swinging movement. FIG. 23 illustrates the encoder 303 mounted as an arc relative to the X-axis. The encoder sensor 302 is disposed at a location opposed to an arc-shaped scaler surface of the linear scale 301, and moves along an arc while maintaining a certain distance from the fixed linear scale 301. Position information read from the encoder 303 is easily substituted by angle information. The relation between a resolution (unit distance dx) of the encoder 303, a basic swing angle dθ, a radius R of a curve is given as dx=R*dθ. When the magnetic linear scale 301 is used, a width of the scale is set to a value considering a range of the swinging movements orthogonal to each other, using a rubberized magnet having certain flexibility and a north pole and a south pole disposed at a pitch of several millimeters, for example.

With the above-described mechanical mechanism, when dynamic tracking irradiation is performed at each radiation node while the X-ray head 100 is continuously moved, stable radiation can be performed with accuracy. Further, with the employment of a robot manipulator which is commonly used in factory automation, quick and high accurate radiation emission becomes possible.

The present disclosure is not limited to the above-described embodiments. Thus, numerous additional modifications and variations are possible in light of the above teachings. The robot manipulator is not limited to six-axis one, but a robot having six axes or more, such as seven-axis manipulator, may be employed. The radiation head is not limited to the X-ray head, but may be a radiation head that uses gamma rays or uncharged particle rays.

The present disclosure can be also applied to laser treatment using carbon dioxide (CO₂) laser or argon (Ar) laser, in addition to radiation therapy. Further, the present disclosure can be widely utilized for a non-destructive testing device, a dynamic tracking sensor, or the like. Furthermore, the present disclosure can be applied to spot operation performed by a moving assembly robot or welding robot.

The above-described embodiments are illustrative and do not limit this disclosure. Thus, numerous additional modifications and variations are possible in light of the above teachings. For example, elements or features of different illustrative and embodiments herein may be combined with or substituted for each other within the scope of this disclosure and the appended claims. Further, features of components of the embodiments, such as number, position, and shape, are not limited to those of the disclosed embodiments and thus may be set as preferred. Further, the above-described steps are not limited to the order disclosed herein. It is therefore to be understood that, within the scope of the appended claims, this disclosure may be practiced otherwise than as specifically described herein.

What is claimed is:

1. A radiation therapy apparatus, comprising:
a manipulator including an arm movable about n axes (where n is a natural number of 6 or more);
a radiation head to emit radiation, supported by the arm of the manipulator;
a correction unit to continuously correct positional coordinates of the radiation head on the manipulator so that radiation emitted from the radiation head is directed onto a reference point while the radiation head is moved along a predetermined route on a virtual sphere having the reference point as its center;
a first controller to control the manipulator so that the radiation head is moved along the route using the corrected positional coordinates; and
a second controller to cause the radiation head to emit radiation toward the reference point as the radiation head passes through radiation emission points on the route.

2. The radiation therapy apparatus according to claim 1, further comprising a memory for storing a plurality of correction values corresponding to a plurality of directions for each of the radiation emission points,
wherein the first controller refers to the memory and continuously moves the radiation head along the route on the virtual sphere using a correction value selected according to a direction of movement of the radiation head on the virtual sphere.

3. The radiation therapy apparatus according to claim 1, further comprising a memory for storing a preset standard route and speed information associated with the standard route,
wherein the first controller corrects a position of the radiation head at the radiation emission points while moving the radiation head along the standard route at a speed specified by the speed information.

4. The radiation therapy apparatus according to claim 1, wherein the first controller continuously moves the radiation head so that vibrations occurring in the radiation head are minimized, when the radiation head changes direction along the route.

5. The radiation therapy apparatus according to claim 1, wherein the radiation emission points include:
radiation nodes disposed at intersections of latitudinal lines on the virtual sphere at predetermined angular intervals and longitudinal lines on the virtual sphere at predetermined angular intervals; and
one or more auxiliary radiation nodes disposed between adjacent radiation nodes ,

6. The radiation therapy apparatus according to claim 5, wherein the first controller moves the manipulator so that the radiation head moves along a route having a minimum number of changes in direction and a maximum number of radiation emission points located along any of the latitudinal lines and the longitudinal lines defined at predetermined angular intervals on the virtual sphere.

7. The radiation therapy apparatus according to claim 5, wherein the first controller moves the manipulator so that the radiation head moves along a route having a maximum number of radiation emission points located along any of the latitudinal lines and the longitudinal lines defined at predetermined angular intervals on the virtual sphere,
the route capable of being drawn with a continuous single stroke.

8. The radiation therapy apparatus according to claim 1, wherein the first controller moves the manipulator so that the radiation head moves along one or more preset standard routes for each treatment region, and
the radiation head emits radiation using at least some of the radiation emission points set on the one or more standard routes.

9. The radiation therapy apparatus according to claim 1, wherein the first controller moves the manipulator so that the radiation head moves along a route having minimum interference between the radiation head and/or the radiation and an external object.

10. The radiation therapy apparatus according to claim 1, wherein the route includes one or more spare nodes, and
the radiation head continues emitting radiation using the one or more spare nodes when radiation emission at any of the radiation emission points is interrupted.

11. The radiation therapy apparatus according to claim 1, wherein the radiation head comprises a collimator device to swing within the radiation head independently of the radiation head, and p1 the collimator device performs dynamic tracking radiation at he radiation emission points while the radiation head is continually moved.

12. A treatment planning device to create a radiation route used by the radiation therapy apparatus according to claim 1, comprising:
a treatment parameter acquisition unit to receive an input of a treatment parameter including a dose distribution;
an image capture unit to capture image information on a treatment region; and
a treatment plan creation unit to select radiation emission points used for radiation emission among a plurality of radiation nodes and one or more auxiliary radiation nodes disposed between adjacent radiation nodes, based on the treatment parameter and the image information,
the radiation nodes disposed at intersections of latitudinal lines on the virtual sphere at predetermined angular intervals and longitudinal lines on the virtual sphere at predetermined angular intervals.

13. A method for controlling a position of a radiation therapy apparatus including a manipulator and a radiation head, the manipulator including an arm movable about n axes (where n is a natural number of 6 or more), the radiation head configured to emit radiation while being supported by the arm, the method comprising:
continuously correcting positional coordinates of the radiation head supported by the manipulator so that radiation emitted from the radiation head is directed onto a reference point and the radiation head is moved along a route on a virtual sphere having the reference point as its center;
controlling the manipulator so that the radiation head is moved along the route on the virtual sphere using the corrected positional coordinates; and
causing the radiation head to emit radiation head toward the reference point as the radiation head passes through radiation emission points on the route.
